(19) United States
(12) Patent Application Publication
(10) Pub. No.: US 2004/0234499 A1
(43) Pub. Date: Nov. 25, 2004

(54) Asthma-related anti-IL-13 immunoglobulin derived proteins, compositions, methods and uses

(76) Inventors: David Shealy, Downingtown, PA (US); Don Edgar Griswold, North Wales, PA (US); George A. Heavner, Malvern, PA (US); Li Li, Downingtown, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/789,002

(22) Filed: Feb. 27, 2004

Related U.S. Application Data
(63) Continuation-in-part of application No. 10/408,694, filed on Apr. 7, 2003.
(63) Continuation-in-part of application No. 10/280,645, filed on Oct. 25, 2002.
(60) Provisional application No. 60/370,371, filed on Apr. 5, 2002.
(60) Provisional application No. 60/343,717, filed on Oct. 26, 2001.

(51) Int. Cl. A61K 38/19; A61K 38/22; A61K 31/56; A61K 39/395

(52) U.S. Cl. 424/85.1; 424/131.1; 514/12; 514/171

(57) ABSTRACT

The present invention relates to therapeutic methods involving the use of at least one asthma related anti-IL-13 human Ig derived protein, as well as such proteins and isolated nucleic acids that encode at least one asthma related Ig derived protein, vectors, host cells, transgenic animals or plants, and methods of making and using thereof, including pharmceutical compositions, methods and devices.

### RELATED APPLICATIONS

This application claims priority to each of: U.S. Provisional patent Appl. No. 60/370,371, filed Apr. 5, 2002; U.S. Non-provisional patent Appl. No. 10/408,694, filed Apr. 7, 2003 (Attorney Docket No. CEN0291 US), U.S. Provisional patent Appl. No. 60/343,717, filed Oct. 26, 2001, and U.S. Non-provisional patent Appl. No. 10/280,645, filed Oct. 25, 2002 (Attorney Docket No. CEN0320 US), each of which is entirely incorporated herein by reference.

### BACKGROUND OF THE INVENTION

1. Field of the Invention

The present invention relates to therapeutic methods involving the use of asthma related anti-interleukin-13 (IL-13) immunoglobulin (Ig) derived proteins, as well as such proteins, asthma-related Ig derived protein encoding and complementary nucleic acids, vectors, host cells, transgenic animals and plants, and methods of making and using thereof, including pharmaceutical compositions, methods and devices.

2. Related Art

Asthma is a chronic inflammation of the bronchial tubes (airways) that causes swelling and narrowing (constriction) of the airways. The result is difficulty breathing. The bronchial narrowing is usually either totally or at least partially reversible with treatments. Bronchial tubes that are chronically inflamed may become overly sensitive to allergens (specific triggers) or irritants (non-specific triggers). The airways may become “twitchy” and remain in a state of heightened sensitivity. This is called “Bronchial Hyperreactivity” (BHR). It is likely that there is a spectrum of bronchial hyperreactivity in all individuals. However, it is clear that asthmatics and allergic individuals (without apparent asthma) have a greater degree of bronchial hyperreactivity than non-asthmatic and non-allergic people. In sensitive individuals, the bronchial tubes are more likely to swell and constrict when exposed to triggers such as allergens, tobacco smoke, or exercise. Amongst asthmatics, some may have mild BHR and no symptoms while others may have severe BHR and chronic symptoms.

Asthma causes a narrowing of the breathing airways, which interferes with the normal movement of air in and out of the lungs. Asthma involves only the bronchial tubes and does not affect the air sacs or the lung tissue. The narrowing that occurs in asthma is caused by three major factors; inflammation, bronchospasm, and hyper-reactivity. The first and most important factor causing narrowing of the bronchial tubes is inflammation. The bronchial tubes become red, irritated, and swollen. The inflammation occurs in response to an allergen or irritant and results from the action of chemical mediators (histamine, leukotrienes, and others). The inflamed tissues produce an excess amount of “sticky” mucus into the tubes. The mucus can clump together and form “plugs” that can clog the smaller airways. Eosinophils and other cells, which accumulate at the site, cause tissue damage. These damaged cells are shed into the airways, thereby contributing to the narrowing.

The muscles around the bronchial tubes tighten during an attack of asthma. This muscle constriction of the airways is called bronchospasm. Bronchospasm causes the airway to narrow further. Chemical mediators and nerves in the bronchial tubes cause the muscles to constrict. In patients with asthma, the chronically inflamed and constricted airways become highly sensitive, or reactive, to triggers such as allergens, irritants, and infections. Exposure to these triggers may result in progressively more inflammation and narrowing. The combination of these three factors results in difficulty with breathing out, or exhaling. As a result, the air needs to be forcefully exhaled to overcome the narrowing, thereby causing the typical “wheezing” sound. People with asthma also frequently “cough” in an attempt to expel the thick mucus plugs. Reducing the flow of air may result in less oxygen passing into the blood stream and if very severe, carbon dioxide may dangerously accumulate in the blood.

Inflammation, or swelling, is a normal response of the body to injury or infection. The bloodflow increases to the affected site and cells rush in and ward off the offending problem. The healing process has begun. Usually, when the healing is complete, the inflammation subsides. Sometimes, the healing process causes scarring. The central issue in asthma, however, is that the inflammation does not resolve completely on its own. In the short term, this results in recurrent “attacks” of asthma. In the long term, it may lead to permanent thickening of the bronchial walls, called airway “remodeling.” If this occurs, the narrowing of the bronchial tubes may become irreversible and poorly responsive to medications. Therefore, the goals of asthma treatment are: (1) in the short term, to control airway inflammation in order to reduce the reactivity of the airways; and (2) in the long term, to prevent airway remodeling.

Asthma symptoms may be activated or aggravated by many agents. Not all asthmatics react to the same triggers. Additionally, the effect that each trigger has on the lungs varies from one individual to another. In general, the severity of your asthma depends on how many agents activate your symptoms and how sensitive your lungs are to them. Most of these triggers can also worsen nasal or eye symptoms. Triggers fall into two categories, allergens (“specific”) and non-allergens—mostly irritants (non-“specific”). Once your bronchial tubes (nose and eyes) become inflamed from an allergic exposure, a re-exposure to the offending allergens will often activate symptoms. These “reactive” bronchial tubes might also respond to other triggers, such as exercise, infections, and other irritants. About 80% of children and 50% of adults with asthma also have allergies. Irritants include respiratory infections, such as those caused by viral “colds,” bronchitis, and sinusitis; drugs, such as aspirin, other NSAIDs (nonsteroidal anti-inflammatory drugs), and Beta Blockers (used to treat blood pressure and other heart conditions); tobacco smoke; outdoor factors, such as smog, weather changes, and diesel fumes; indoor factors, such as paint, detergents, deodorants, chemicals, and perfumes; nighttime; GERD (gastro-esophageal reflux disorder); exercise, especially under cold dry conditions; work-related factors, such as chemicals, dusts, gases, and metals; and emotional factors, such as laughing, crying, yelling, and distress; hormonal factors, such as in premenstrual syndrome.

Asthma is often referred to as being “extrinsic” or “intrinsic.” A better understanding of the nature of asthma can help explain the differences between them. Extrinsic, or allergic asthma, is more common (90% of all cases) and typically develops in childhood. Eighty percent of children with asthma also have documented allergies. Typically, there is a family history of allergies. Additionally, other allergic conditions, such as hay fever or eczema, are often also present. Allergic asthma often goes into remission in early adulthood. However, in 75% of cases, the asthma reappears later. Intrinsic asthma represents about 10% of all cases. It usually develops after the age of 30 and is not typically associated with allergies. Women are more frequently involved and many cases seem to follow a respiratory tract infection. The condition can be difficult to treat and symptoms are often chronic and year-round.

The symptoms of asthma vary from person to person and in any individual from time to time. It is important to remember that many of these symptoms can be subtle and similar to those seen in other conditions. All of the symptoms mentioned below can be present in other respiratory, and sometimes, in heart conditions. This potential confusion makes identifying the settings in which the symptoms occur and diagnostic testing very important in recognizing this disorder. The four major recognized symptoms include, (1) shortness of breath (especially with exertion or at night); (2) wheezing (a whistling or hissing sound when breathing out); (3) coughing (may be chronic; usually worse at night and early morning and may occur after exercise or when exposed to cold, dry air); and (4) chest tightness (which may occur with or without the above symptoms). Asthma is classified according to the frequency and severity of symptoms, or “attacks,” and the results of pulmonary (lung) function tests: 30% of affected patients have mild, intermittent (less than 2 episodes a week) symptoms of asthma with normal breathing tests; 30% have mild, persistent (2 or mores episodes a week) symptoms of asthma with normal breathing tests; and 40% have moderate or severe, persistent (daily or continuodsus) symptoms of asthma with abnormal breathing tests.

Most asthma medications work by relaxing bronchospasm (bronchodilators) or reducing inflammation (corticosteroids). In the treatment of asthma, inhaled medications are generally preferred over tablet or liquid medicines that are swallowed (oral medications). Inhaled medications act directly on the airway surface and airway muscles where the asthma problems initiate. Absorption of inhaled medications into the rest of the body is minimal. Therefore, adverse side effects are fewer as compared to oral medications. Inhaled medications include beta-2 agonists, anticholinergics, corticosteroids, and cromolyn sodium. Oral medications include aminophylline, and corticosteroid tablets.

Interleukin 13 (IL-13) is a pleiotropic cytokine mainly produced by Th2 cells, and exhibits a variety of effects that may be relevant to asthma, allergy and other Th2 dominated responses. IL-13 induces IgE production, CD23 up regulation, VCAM-1 expression and directly stimulates eisoinphils and mast cells.

Non-human, chimeric, polyclonal (e.g., anti-sera) and/or monoclonal antibodies (Mabs) and fragments (e.g., proteolytic digestion products thereof) are potential therapeutic agents that are being developed in some cases to attempt to treat certain diseases. However, such antibodies that comprise non-human portions elicit an immune response when administered to humans. Such an immune response can result in an immune complex-mediated clearance of the antibodies from the circulation, and make repeated administration unsuitable for therapy, thereby reducing the therapeutic benefit to the patient and limiting the readministration of the Ig derived protein. For example, repeated administration of antibodies comprising non-human portions can lead to serum sickness and/or anaphalaxis. In order to avoid these and other such problems, a number of approaches have been taken to reduce the immunogenicity of such antibodies and portions thereof, including chimerization and “humanization,” as well known in the art. These approaches have produced antibodies having reduced immunogenicity, but with other less disirable properties.

Accordingly, there is a need to provide asthma related antibody, receptor and antibody fusion or related proteins, nucleic acids, host cells, compositions, and methods of making and using thereof, that overcome one more of these problems, as well as improvements over known human or humanized asthma related protein antibodies, antibody fusion proteins, or variants thereof.

### SUMMARY OF THE INVENTION

The present invention provides isolated asthma related immunoglobulin (Ig) derived proteins (Ig derived proteins), including antibodies, immunoglobulins, receptor fusion proteins, cleavage products and other specified portions and variants thereof, as well as asthma related Ig derived protein compositions, encoding or complementary nucleic acids, vectors, host cells, compositions, formulations, devices, transgenic animals, transgenic plants, and methods of making and using thereof, as described and enabled herein, in combination with what is known in the art. Such asthma related Ig derived proteins act as antagonists to asthma related proteins and thus are useful for treated asthma related pathologies.

The present invention provides at least one method for treating an asthma related condition in a cell, tissue, organ or animal, comprising contacting or administering an asthma modulating effective amount of at least one asthma related human Ig derived protein with, or to, said cell, tissue, organ or animal, optionally wherein said animal is a primate, optionally a monkey or a human. The method can further include where said asthma related condition is at least one selected from asthma, emphysema, asthma, chronic bronchitis or airflow obstruction, or optionally wherein said effective amount is 0.01-100 mg/kilogram of said cells, tissue, organ or animal. Such a method can further include wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

Such a method can further comprise administering, prior, concurrently or after said (a) contacting or administering, at least one composition comprising a therapeutically effective amount of at least one compound or protein selected from at least one of inhaled asthma medication, such as but not limited to an asthma related therapeutic, a TNF antagonist, an antirheumatic, a muscle relaxant, a narcotic, an analgesic, an anesthetic, a sedative, a local anethetic, a neuromuscular blocker, an antimicrobial, an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

The present invention further provides at least one asthma related Ig derived protein, specified portion or variant in a method or composition, when administered in a therapeutically effective amount, for modulation, for treating or reducing the symptoms of asthma and related disorders, such as asthma, associated pulmonary or sinus inflammation leading to at least one of inspatory or expatory wheezing, breathlessness, chest tightness, coughing, dyspnea, burning, airway edema, excess mucus, bronchospasm, tachypnea, tachycardia, cyanosis, allergic rhinitis, infections (e.g., fungal or bacterial), and the like, as needed in many different conditions, such as but not limited to, prior to, subsequent to, or during an asthma related disease treatment, as known in the art.

The present invention also provides at least one isolated asthma related Ig derived protein, comprising at least one immnuoglobulin complementarity determining region (CDR) or at least one ligand binding region (LBR) that specifically binds at least one asthma related protein, wherein (a) said asthma related Ig derived protein specifically binds at least one epitope comprising at least 1-3, to the entire amino acid sequence of at least one asthma related protein selected from the group consisting of a human interleukin-13 (e.g., but not limited to, SEQ ID NO:1 or muteins thereof (e.g., R130Q, Ile48, Val48, Gln90, Glu90, Leu95, Ile95, Leu96, Ile96, Leu99, Ile99, Phe103, Tyr103, Asn130 and/or Gln130), as 1-145 amino acids, such as but not limited to at least one of 1-10, 10-20, 20-30, 30-40, 40-50, 50-60, 60-70, 70-80, 80-90, 90-100, 100-110, 110-120, 120-130, 130-140, and/or 14-145 of SEQ ID NO:1; said asthma related Ig derived protein optionally binding asthma related protein such as IL-13 with an affinity of at least 10⁻⁹ M, at least 10⁻¹¹ M, or at least 10⁻¹² M; said human Ig derived optionally and substantially neutralizes at least one activity of at least one asthma related protein or hormone.

The invention also provides at least one method for producing at least one asthma related human Ig derived protein, comprising translating such a nucleic acid or an endogenous nucleic acid that hybridizes thereto under stringent conditions, under conditions in vitro, in vivo or in situ, such that the asthma related human Ig derived protein is expressed in detectable or recoverable amounts.

The invention also provides at least one asthma related human Ig derived protein composition, comprising at least one isolated asthma related human Ig derived protein and a carrier or diluent, optionally further wherein said carrier or diluent is pharmaceutically acceptable, and/or further comprising at least one compound or protein selected from inhaled asthma medication such as but not limited to an asthma related therapeutic, a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

The present invention also provides at least one medical device, comprising at least one asthma related human Ig derived protein, wherein said device is suitable to contacting or administerting said at least one asthma related human Ig derived protein by at least one mode selected from parenteral, subcutaneous, intramuscular, intravenous, intrarticular, intrabronchial, intraabdoninal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

The invention also includes at least one formulation comprising at least one asthma related human Ig derived protein, and at least one selected from sterile water, sterile buffered water, or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent, optionally wherein the concentration of asthma related human Ig derived protein is about 0.1 mg/ml to about 100 mg/ml, further comprising at least one isotonicity agent or at least one physiologically acceptable buffer.

The invention further provides at least one method for preparing a formulation of at least one asthma related human Ig derived protein of the invention, comprising admixing at least one asthma related human Ig derived protein in at least one buffer containing saline or a salt.

The invention also provides at least one method for producing at least one asthma related human Ig derived protein of the invention, comprising providing a host cell, transgenic animal, transgenic plant or plant cell capable of expressing in recoverable amounts said human Ig derived protein, optionally further wherein said host cell is a mammalian cell, a plant cell or a yeast cell; said transgenic animal is a mammal; said transgenic mammal is selected from a goat, a cow, a sheep, a horse, and a non-human primate.

The present invention further provides any invention described herein and is not limited to any particular description, embodiment or example provided herein.

### DESCRIPTION OF THE INVENTION

The present invention provides therapeutic methods comprising administering an isolated, recombinant and/or synthetic asthma related Ig derived protein. Such Ig derived proteins of the present invention comprise specific Ig derived protein sequences, domains, fragments and specified variants thereof, and methods of making and using thereof, including therapeutic compositions, methods and devices.

As used herein, “asthma related Ig derived protein,” and the like, modulates, affects, antagonizes, decreases, blocks, inhibits, abrogates, enhances, agonizes, or interferes with at least one asthma related protein activity, binding or asthma related protein receptor activity or binding in vitro, in situ and/or preferably in vivo. For example, a suitable asthma related Ig derived protein, specified portion or variant of the present invention can bind at least one asthma related protein or receptor and includes asthma related Ig derived proteins, antigen-binding fragments thereof, and specified portions, variants or domains thereof that bind specifically to asthma related. A suitable asthma related Ig derived protein, specified portion, or variant can also modulates, affects, antagonizes, decreases, blocks, inhibits, abrogates, enhances, agonizes, or interferes with at least one asthma related protein RNA, DNA or protein synthesis, asthma related protein release, asthma related protein or receptor signaling, membrane asthma related protein cleavage, asthmaprotein related activity, asthma related protein production and/or synthesis, e.g., as described herein or as known in the art. In a preferred embodiment, the asthma related protein is human interleukin-13 (IL-13).

Asthma related Ig derived proteins useful in the methods and compositions of the present invention are characterized by high affinity binding to asthma related proteins, and optionally and preferably having low toxicity. In particular, an Ig derived protein, specified fragment or variant of the invention, where the individual components, such as the variable region, constant region and/or framework, individually and/or collectively, optionally and preferably possess low immunogenicity, is useful in the present invention. The Ig derived proteins that can be used in the invention are optionally characterized by their ability to treat patients for extended periods with good to excellent alleviation of symptoms and low toxicity. Low immunogenicity and/or high affinity, as well as other suitable properties, may contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), each of the above references entirely incorporated herein by reference.)

Utility

The isolated nucleic acids of the present invention can be used for production of at least one asthma related Ig derived protein can be used to effect in an cell, tissue, organ or animal (including mammals and humans), to modulate, treat, alleviate, help prevent the incidence of, or reduce the symptoms of, at least one asthma related pathology, disease, condition or symptom.

Such a method can comprise administering an effective amount of a composition or a pharmaceutical composition comprising at least one asthma related Ig derived protein or specified portion or variant to a cell, tissue, organ, animal or patient in need of such modulation, treatment, alleviation, prevention, or reduction in symptoms, effects or mechanisms. The effective amount can comprise an amount of about 0.001 to 100 mg/kg per single or multiple administration, or to achieve a serum concentration of 0.01-5000 μg/ml serum concentration per single or multiple adminstration, or any effective range or value therein, as done and determined using known methods, as described herein or known in the relevant arts.

Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., New York, N.Y. (1987-2004); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., N.Y. (1994-2004); Colligan et al., Current Protocols in Protein Science, John Wiley & Sons, New York, N.Y., (1997-2004).

Immunoglobulin (Ig) Derived Proteins of the Present Invention

The term “immunoglobulin derived protein” or “Ig derived protein” is intended to encompass Ig derived proteins, digestion fragments, specified portions and variants thereof, including Ig derived protein mimetics or comprising portions of Ig derived proteins that mimic the structure and/or function of an anitbody or specified fragment or portion thereof, including single chain Ig derived proteins and fragments thereof, and is also is intended to encompass proteins that contain mimetics to therapeutic proteins, antibodies, and digestion fragments, specified portions and variants thereof, wherein the protein comprises at least one functional asthma related protein ligand binding region (LBR) that optionally replaces at least one complementarity determing region (CDR) of the antibody from which the Ig-derived protein, portion or variant is derived. Such asthma related Ig derived proteins, specified portions or variants include those that mimic the structure and/or function of at least one asthma related protein antagonist, such as an asthma related protein antibody or receptor or ligand protein, or fragment or analog. Functional fragments include antigen-binding fragments that bind to asthma related proteins or fragments thereof. For example, Ig derived protein fragments capable of binding to human asthma related proteins or fragments thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Ig derived proteins can also be produced in a variety of truncated forms using Ig derived protein genes in which one or more stop codons have been introduced upstream of the natural stop site. For example, a chimeric gene encoding a F(ab′)₂ heavy chain portion can be designed to include DNA sequences encoding the CH₁ domain and/or hinge region of the heavy chain. The various portions of Ig derived proteins can be joined together chemically by conventional techniques, or can be prepared as a contiguous protein using genetic engineering techniques. For example, a nucleic acid encoding the variable and constant regions of a human Ig derived protein chain can be expressed to produce a contiguous protein. See, e.g., Colligan, Current Protocols in Immunology, supra, sections 2.8 and 2.10, for fragmentation, and Ladner et al., U.S. Pat. No. 4,946,778 and Bird, R. E. et al., Science, 242: 423426 (1988), regarding single chain Ig derived proteins, each of which publications are entirely incorporated herein by reference.

As used herein, the term “human Ig derived protein” refers to an Ig derived protein in which substantially every part of the protein (e.g., CDR, LBR, framework, C_{L}, C_{H} domains (e.g., C_{H}1, C_{H}2, C_{H}3), hinge, (V_{L}, V_{H})) is substantially non-immunogenic, with only minor sequence changes or variations. Such changes or variations optionally and preferably retain or reduce the immunogenicity in humans relative to non-modified human Ig derived proteins. Thus, a human Ig derived protein is distinct from a chimeric or humanized Ig. It is pointed out that a human Ig derived protein can be produced by a non-human animal or prokaryotic or eukaryotic cell that is capable of expressing functionally rearranged human immunoglobulin (e.g., heavy chain and/or light chain) genes. Further, when a human Ig derived protein is a single chain Ig derived protein, it can comprise a linker peptide that is not found in native human Ig derived proteins. For example, an Fv can comprise a linker peptide, such as two to about eight glycine or other amino acid residues, which connects the variable region of the heavy chain and the variable region of the light chain. Such linker peptides are considered to be of human origin. Asthma related Ig derived proteins that comprise at least one asthma related protein ligand or receptor thereof can be designed against an appropriate ligand, such as isolated and/or asthma related protein, or a portion thereof (including synthetic molecules, such as synthetic peptides). Preparation of such asthma related Ig derived proteins are performed using known techniques to identify and characterize ligand binding regions or sequences of at least one asthma related protein or portion thereof.

Ig derived proteins that are specific for an asthma related protein can be raised against an appropriate immunogenic antigen, such as isolated and/or asthma related protein or a portion thereof (including synthetic molecules, such as synthetic peptides). Preparation of immunogenic antigens, and monoclonal Ig derived protein production can be performed using any suitable technique. A variety of methods have been described (see e.g., Harlow and Lane, Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004); Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Current Protocols In Molecular Biology, Ausubel, F. M. et al., Eds., (John Wiley & Sons: New York, N.Y.), Chapter 11, (1991-2004)), each of which is entirely incorporated herein by reference. Generally, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art, see, e.g., www.atcc.org, www.lifetech.com., and the like, each of which is entirely incorporated herein by reference with Ig derived protein producing cells, such as, but not limited to, isolated or cloned spleen cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like, or any combination thereof. See, e.g., Ausubel, supra, and Colligan, Immunology, supra, chapter 2, each entirely incorporated herein by reference.

Ig derived protein producing cells can be obtained from the peripheral blood or, preferably the spleen or lymph nodes, of mice, rats, rabbits, primates, such as humans, or other suitable animals that have been immunized with the antigen of interest, including boosting with antigen or a nucleic acid encoding such such antigen. Any other suitable host cell can also be used for expressing heterologous or endogenous nucleic acid encoding an Ig derived protein, specified fragment or variant thereof, of the present invention. The fused cells (e.g., hybridomas) or recombinant cells can be isolated using selective culture conditions or other suitable known methods, and cloned by limiting dilution or cell sorting, or other known methods. Cells which produce Ig derived proteins with the desired specificity can be selected by a suitable assay (e.g., ELISA). See., e.g., Ausubel, et al., ed., Current Protocols in Molecular Biology, John Wiley & Sons, Inc., New York, N.Y. (1987-2004); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), each which is entirely incorporated herein by reference.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, Del; Biovation, Aberdeen, Scotland, UK; Biolnvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350260(May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (Biolnvent); WO88/06630; WO90/3809 (Dyax); U.S. Pat. No. 4,704,692 (Enzon); PCT/US91/02989 (Affymax); WO89/06283; EP 371/998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, now Applied Molecular Evolution (AME), Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), (each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (Nov. 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. U.S. Pat. No. 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)), each entirely incorporated herein by reference.

Methods for humanizing non-human Ig derived proteins can also be used and are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following methods such, but not limited to, Jones et al., Nature 321:522 (1986); Riechmann et al., Nature 332:323 (1988); Verhoeyen et al., Science 239:1534 (1988); Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), each of which is entirely incorporated herein by reference), by substituting rodent CDRs or CDR sequences for the corresponding sequences of a human antibody. Accordingly, such “humanized” Ig derived proteins can be chimeric Ig derived proteins, wherein substantially less than an intact human variable domain has been substituted by the corresponding sequence from a non-human species. In practice, humanized Ig derived proteins are typically human Ig derived proteins in which some CDR residues and possibly some FR residues are substituted by residues from analogous sites in rodent Ig derived proteins, where the FR residues may be needed to retain, maintain, enhance, or modify binding activity, such as, but not limited to, specificity, affinity, avidity, on-rate, off-rate, and the like, as known in the art and/or as taught herein.

The choice of human variable domains, both light and heavy, to be used in making the humanized Ig derived proteins can be used to affect binding activity or half-life, or reduce immunogenicity. As a non-limiting example, according to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol. 151: 2296 (1993); Chothia and Lesk, J. Mol. Biol. 196:901 (1987); Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), each of which is entirely incorporated herein by reference). Another method uses a particular framework derived from the consensus sequence of all human Ig derived proteins of a particular subgroup of light or heavy chains. The same framework can be used for several different humanized Ig derived proteins (Carter et al., Proc. Natl. Acad. Sci. U.S.A. 89:4285 (1992); Presta et al., J. Immunol. 151:2623 (1993), each of which is entirely incorporated herein by reference).

Ig derived proteins can also optionally be humanized with retention of binding activity for the antigen and other favorable or desired biological properties. To achieve this goal, according to a preferred method, humanized Ig derived proteins are prepared by a process of analysis of the parental sequences and various conceptual humanized products using three-dimensional models of the parental and humanized sequences. Three-dimensional immunoglobulin models are commonly available and are familiar to those skilled in the art. Computer programs are known in the art that analyze and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as but not limited to increased affinity for the target antigen(s), is achieved. In general, the CDR residues can be directly and substantially involved in influencing antigen binding, but FR sequences can also influence the binding activity of the Ig derived protein.

Monoclonal Ig derived proteins can be made by the hybridoma method. Human myeloma and rodent-rodent or rodent-human heteromyeloma cell lines for the production of human monoclonal Ig derived proteins have been described, for example, by Kozbor, J. Immunol. 133:3001 (1984); Brodeur et al., Monoclonal Antibody Production Techniques and Applications, pp. 51-63 (Marcel Dekker, Inc., New York, 1987); and Boerner et al., J. Immunol. 147:86 (1991); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), each of which is entirely incorporated herein by reference.

Alternatively, phage display technology, e.g., as presented above, can be used to produce human Ig derived proteins and antibody fragments in vitro, from immunoglobulin variable (V) domain gene repertoires from unimmunized donors. According to one none limiting example of this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson et al., Current Opinion in Structural Biology 3:564 (1993), each of which is entirely incorporated herein by reference. Several sources of V-gene segments can be used for phage display. Clackson et al., Nature 352:624 (1991) isolated a diverse array of anti-oxazolone Ig derived proteins from a small random combinatorial library of V genes derived from the spleens of immunized mice. A repertoire of V genes from unimmunized human donors can be constructed and Ig derived proteins to a diverse array of antigens (including self-antigens) can be isolated essentially following the techniques described by Marks et al., J. Mol. Biol. 222:581 (1991), or Griffith et al., EMBO J. 12:725 (1993); Antibodies, a Laboratory Manual, Cold Spring Harbor, N.Y. (1989); Colligan, et al., eds., Current Protocols in Immunology, John Wiley & Sons, Inc., NY (1994-2004); Colligan et al., Current Protocols in Protein Science, John Wiley & Sons, New York, N.Y., (1997-2004), each of which is entirely incorporated herein by reference.

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10:779 (1992)). In this method, the affinity of “primary” human Ig derived proteins obtained by phage display can be improved by sequentially replacing the heavy and light chain V region genes with repertoires of naturally occurring variants (repertoires) of V domain genes obtained from unimmunized donors. This technique allows the production of Ig derived proteins and antibody fragments with affinities in the nM range, e.g., 10×10⁻⁶ to 10×10⁻¹³ M. A strategy for making very large phage antibody repertoires has been described, as a non-limiting example, by Waterhouse et al., Nucl. Acids Res. 21:2265 (1993). Gene shuffling can also be used to derive human Ig derived proteins from rodent Ig derived proteins, where the human antibody has similar affinities and specificities to the starting rodent antibody. According to this method, which is also referred to as “epitope imprinting”, the heavy or light chain V domain gene of rodent Ig derived proteins obtained by phage display technique is replaced with a repertoire of human V domain genes, creating rodent-human chimeras. Selection with antigen results in isolation of human variable capable of restoring a functional antigen-binding site, i.e. the epitope governs (imprints) the choice of partner. When the process is repeated in order to replace the remaining rodent V domain, a human antibody is obtained (see PCT WO 93/06213, published 1 Apr. 1993). Unlike traditional humanization of rodent Ig derived proteins by CDR grafting, this technique provides completely human Ig derived proteins, which have no framework or CDR residues of rodent origin.

According to a different approach, antibody-variable domains with the desired binding specificities (antibody-antigen combining sites) are fused to immunoglobulin constant-domain sequences. The fusion preferably is with an immunoglobulin heavy-chain constant domain, comprising at least part of the hinge, the second heavy chain constant region (C.sub.H2), and the third heavy chain constant region (C.sub.H3). It is preferred to have the first heavy-chain constant region (C.sub.H1), containing the site necessary for light-chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the production of at least two polypeptide chains in equal ratios results in high yields or when the ratios are of no particular significance. In a preferred embodiment of this approach, the bispecific Ig derived proteins are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. For further details of generating bispecific Ig derived proteins, see, for example, Suresh et al., Methods in Enzymology 121:210 (1986).

Heteroconjugate Ig derived proteins are also within the scope of the present invention. Heteroconjugate Ig derived proteins are composed of two covalently joined Ig derived proteins. Such Ig derived proteins have, for example, been proposed to target immune system cells to unwanted cells (U.S. Pat. No. 4,676,980), and for treatment of HIV infection (WO 91/00360; WO 92/00373; and EP 03089). Heteroconjugate Ig derived proteins can be made using any convenient cross-linking methods. Suitable cross-linking agents are well known in the art, and are disclosed in U.S. Pat. No. 4,676,980, along with a number of cross-linking techniques.

At least one asthma related Ig derived protein of the present invention is produced by a cell line, a mixed cell line, an immortalized cell or clonal population of immortalized cells. Immortalized asthma related producing cells can be produced using suitable methods, for example, fusion of a human Ig derived protein-producing cell and a heteromyeloma or immortalization of an activated human B cell via infection with Epstein Barr virus (Niedbala et al., Hybridoma, 17(3):299-304 (1998); Zanella et al., J Immunol Methods, 156(2):205-215 (1992); Gustafsson et al., Hum Ig derived proteins Hybridomas, 2(1)26-32 (1991)). Preferably, the human anti-human asthma related proteins or fragments or specified portions or variants is generated by immunization of a transgenic animal (e.g., mouse, rat, hamster, non-human primate, and the like) capable of producing a repertoire of human Ig derived proteins, as described herein and/or as known in the art. Cells that produce a human anti- human asthma related Ig derived protein can be isolated from such animals and immortalized using suitable methods, such as the methods described herein.

Transgenic mice that can produce a repertoire of human Ig derived proteins that bind to human antigens can be produced by known methods (e.g., but not limited to, U.S. Pat. Nos: 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce Ig derived proteins encoded by endogenous genes.

The term “functionally rearranged,” as used herein refers to a segment of DNA from an immunoglobulin locus that has undergone V(D)J recombination, thereby producing an immunoglobulin gene that encodes an immunoglobulin chain (e.g., heavy chain, light chain), or any portion thereof. A functionally rearranged immunoglobulin gene can be directly or indirectly identified using suitable methods, such as, for example, nucleotide sequencing, hybridization (e.g., Southern blotting, Northern blotting) using probes that can anneal to coding joints between gene segments or enzymatic amplification of immunoglobulin genes (e.g., polymerase chain reaction) with primers that can anneal to coding joints between gene segments. Whether a cell produces an Ig derived protein comprising a particular variable region or a variable region comprising a particular sequence (e.g., at least one CDR sequence) can also be determined using suitable methods. In one example, MRNA can be isolated from an Ig derived protein-producing cell (e.g., a hybridoma or recombinant cell or other suitable source) and used to produce cDNA encoding the Ig derived protein thereof. The cDNA can be cloned and sequenced or can be amplified (e.g., by polymerase chain reactionor other known and suitable methods) using a first primer that anneals specifically to a portion of the variable region of interest (e.g., CDR, coding joint) and a second primer that anneals specifically to non-variable region sequences (e.g., C_{H}1, V_{H}).

Screening Ig derived protein or specified portion or variants for specific binding to similar proteins or fragments can be conveniently achieved using peptide display libraries. This method involves the screening of large collections of peptides for individual members having the desired function or structure. Ig derived protein screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Ig derived protein Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge Ig derived protein Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Ig derived proteins, specified portions and variants of the present invention can also be prepared using at least one asthma related Ig derived protein encoding nucleic acid to provide transgenic animals or mammals, such as goats, cows, horses, sheep, and the like, that produce such Ig derived proteins or specified portions or variants in their milk. Such animals can be provided using known methods. See, e.g., but not limited to, U.S. Pat. Nos. 5,827,690; 5,849,992; 4,873,316; 5,849;992; 5,994,616; 5,565,362; 5,304,489, and the like, each of which is entirely incorporated herein by reference.

Ig derived proteins, specified portions and variants of the present invention can additionally be prepared using at least one asthma related Ig derived protein encoding nucleic acid to provide transgenic plants and cultured plant cells (e.g., but not limited to tobacco and maize) that produce such Ig derived proteins, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Ig derived proteins have also been produced in large amounts from transgenic plant seeds including Ig derived protein fragments, such as single chain Ig derived proteins (scFv's), including tobacco seeds and potato tubers. See, e.g., Conrad et al., Plant Mol. Biol. 38:101-109 (1998) and reference cited therein. Thus, Ig derived proteins, specified portions and variants of the present invention can also be produced using transgenic plants, according to know methods. See also, e.g., Fischer et al., Biotechnol. Appi. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. See, also generally for plant expression of Ig derived proteins, but not limited to, Each of the above references is entirely incorporated herein by reference.

The Ig derived proteins of the invention can bind human asthma related proteins or fragments with a wide range of affinities (K_{D}). In a preferred embodiment, at least one human mAb of the present invention can optionally bind human asthma related proteins or fragments with high affinity. For example, a human mAb can bind human asthma related proteins or fragments with a K_{D} equal to or less than about 10⁻⁸ M or 10⁻⁹ M or, more preferably, with a K_{D} equal to or less than about 0.1-9.9 (or any range or value therein) X 10⁻¹⁰ M, 10⁻¹¹, 10⁻¹², 10⁻¹³ or any range or value therein.

The affinity or avidity of an Ig derived protein for an antigen can be determined experimentally using any suitable method. (See, for example, Berzofsky, et al., “Ig derived protein-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular Ig derived protein-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of Ig derived protein and antigen, and a standardized buffer, such as the buffer described herein.

Nucleic Acid Moleculess

Using the information provided herein, such as the nucleotide sequences encoding at least 90-100% of the contiguous amino acids of at least one of asthma related Ig derived protein of the present invention, specified fragments, variants or consensus sequences thereof, or a deposited vector comprising at least one of these sequences, a nucleic acid molecule of the present invention encoding at least one asthma related Ig derived protein or specified portion or variant, can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, e.g., but not limited to, at least one specified portion of at least one CDR, as CDR1, CDR2 and/or CDR3 of at least one heavy chain or light chain, respectively; nucleic acid molecules comprising the coding sequence for an asthma related Ig derived protein or specified portion or variant; and nucleic acid molecules which comprise a nucleotide sequence substantially different from those described above but which, due to the degeneracy of the genetic code, still encode at least one asthma related Ig derived protein as described herein and/or as known in the art. Of course, the genetic code is well known in the art. Thus, it would be routine for one skilled in the art to generate such degenerate nucleic acid variants that code for specific asthma related Ig derived protein or specified portion or variants of the present invention. See, e.g., Ausubel, et al., supra, and such nucleic acid variants are included in the present invention.

In another aspect, the invention provides isolated nucleic acid molecules encoding a(n) asthma related Ig derived protein having an amino acid sequence as encoded by the nucleic acid contained in the plasmid deposited as designated clone names ______ and ATCC Deposit Nos. ______, respectively, deposited on ______.

As indicated herein, nucleic acid molecules of the present invention which comprise a nucleic acid encoding an asthma related Ig derived protein can include, but are not limited to, those encoding the amino acid sequence of an Ig derived protein fragment, by itself; the coding sequence for the entire Ig derived protein or a portion thereof; the coding sequence for an Ig derived protein, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an Ig derived protein can be fused to a marker sequence, such as a sequence encoding a peptide that facilitates purification of the fused Ig derived protein comprising an Ig derived protein fragment or portion.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 90-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. No. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences. See, e.g., Ausubel, supra, Colligan, supra.

Recombinant Expression Cassettes

The present invention further provides recombinant expression cassettes comprising a nucleic acid of the present invention. A nucleic acid sequence of the present invention, for example a cDNA or a genomic sequence encoding an Ig derived protein of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

A polynucleotide of the present invention can be expressed in either sense or anti-sense orientation as desired. It will be appreciated that control of gene expression in either sense or anti-sense orientation can have a direct impact on the observable characteristics. Another method of suppression is sense suppression. Introduction of nucleic acid configured in the sense orientation has been shown to be an effective means by which to block the transcription of target genes.

A variety of cross-linking agents, alkylating agents and radical generating species as pendant groups on polynucleotides of the present invention can be used to bind, label, detect and/or cleave nucleic acids. Knorre, et al., Biochimie 67:785-789 (1985); Vlassov, et al., Nucleic Acids Res. 14:4065-4076 (1986); Iverson and Dervan, J. Am. Chem. Soc. 109:1241-1243 (1987); Meyer, et al., J. Am. Chem. Soc. 111:8517-8519 (1989); Lee, et al., Biochemistry 27:3197-3203 (1988); Home, et al., J. Am. Chem. Soc. 112:2435-2437 (1990); Webb and Matteucci, J. Am. Chem. Soc. 108:2764-2765 (1986); Nucleic Acids Res. 14:7661-7674 (1986); Feteritz, et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681941, each entirely incorporated herein by reference.

Vectors And Host Cells

The present invention also relates to vectors that include isolated nucleic acid molecules of the present invention, host cells that are genetically engineered with the recombinant vectors, and the production of at least one asthma related Ig derived protein by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat.Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one Ig derived protein of the present invention can be expressed in a modified form, such as a fusion protein, and can include not only secretion signals, but also additional heterologous functional regions. For instance, a region of additional amino acids, particularly charged amino acids, can be added to the N-terminus of an Ig derived protein to improve stability and persistence in the host cell, during purification, or during subsequent handling and storage. Also, peptide moieties can be added to an Ig derived protein of the present invention to facilitate purification. Such regions can be removed prior to final preparation of an Ig derived protein or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Alternatively, nucleic acids of the present invention can be expressed in a host cell by turning on (by manipulation) in a host cell that contains endogenous DNA encoding an Ig derived protein of the present invention. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the Ig derived proteins, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610, DG-44) and BSC-1 (e.g., ATCC CRL-26) cell lines, HepG2 cells, P3X63Ag8.653, SP/0, NSO, NS1, NS2, AE-1, L.5, SP2/0-Ag14, 293, MLA 144, ACT IV, DA-1, JURKAT, WEHI, TAJI, NIH 3T3, Sf-9, PerC.6, YB2/0, NAMAIWA, NEURO-2A, or HeLa cells and the like, which are readily available from, for example, American Type Culture Collection, Manassas, Va. Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a preferred embodiment, the recombinant cell is a P3X63Ab8.653 or a SP2/0-Agl4 cell.

Expression vectors for these cells can include one or more of the following expression control sequences, such as, but not limited to an origin of replication; a promoter (e.g., late or early SV40 promoters, an early or late hCMV promoter (e.g., U.S. Pat. Nos. 5,168,062; 5,385,839), an HSV tk promoter, a pgk (phosphoglycerate kinase) promoter, an EF-1 alpha promoter (e.g., U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. A(n) example of a terminator sequence is the polyadenlyation sequence from the bovine growth hormone gene. Sequences for accurate splicing of the transcript can also be included. A(n) example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Ig Derived Protein or Specified Portion or Variant Thereof

An asthma related Ig derived protein can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See e.g., Colligan, Current Protocols in Immunology, or Current Protocols in Protein Science, John Wiley & Sons, New York, N.Y., (1997-2004), e.g., Chapters 1, 4, 6, 8, 9, 10, each entirely incorporated herein by reference.

Ig derived proteins of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the Ig derived protein of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Asthma Relatedf Ig Derived Proteins, Fragments and/or Variants

The isolated Ig derived proteins of the present invention comprise an Ig derived protein encoded by any one of the polynucleotides of the present invention as discussed more fully herein, or any isolated or prepared Ig derived protein thereof. Preferably, the human Ig derived protein or antigen-binding fragment binds human asthma related proteins or fragments and, thereby substantially neutralizes the biological activity of the protein. A(n) Ig derived protein that partially or preferably substantially neutralizes at least one biological activity of at least one asthma related protein or fragment can bind the protein or fragment and thereby inhibit activities mediated through the binding of asthma related to the asthma related receptor or through other asthma related-dependent or mediated mechanisms. As used herein, the term “neutralizing Ig derived protein” refers to an Ig derived protein that can inhibit human asthma related protein or fragment related-dependent activity by about 20-120%, preferably by at least about 60, 70, 80, 90, 91, 92, 93, 94, 95, 96, 97, 98, 99, 100% or more depending on the assay.

Asthma Related Protein Assays. The capacity of anti-human asthma related Ig derived protein to inhibit human asthma related related-dependent activity is preferably assessed by at least one suitable asthma assay, as described herein and/or as known in the art. Asthma related assays include, but are not limited to, inhibition of at least one of airway hyperresponsiveness (AHR), goblet cell hyperplasia and/or mucus production in B9 cells in vitro or in situ; and inhibition of at least one of the production of IL-5, IL-6, eotaxin, KC, MIP-1 and MCP-1 in the lung, in vitro, in vivo, or in situ, e.g., as presented in Example 2, below, and as known in the art. See, e.g., www.copewithcytokines.de, with reference to IL-13 and IL-13 bioassays and referenences cited therein (e.g., but not limited to, as presented in Mire-Sluis and Thorpe “Laboratory protocols for the quantitation of cytokines by bioassay using cytokine responsive cell lines.” J.Immunol. Meth. 211(1-2):199-210 (1998); Wadhwa and Thorpe, “Cytokine immunoassays: recommendations for standardisation, calibration and validation.” J. Immunol. Meth. 219(1-2): 1-5 (1998); Walker et al., “Enzyme-labeled antibodies in bioassays.” Meth. Biochem. Anal. 36:179-208 (1992); Whiteside, “Cytokine measurements and interpretation of cytokine assays in human disease.” J. Clin. Immunol. 14(6):327-339 (1994); Bienvenu et al., “Cytokine assays in human sera and tissues.” Toxicol.129(1):55-61 (1998)), which are each entirely incorporated herein by reference.

Asthma Related Ig Derived Protein Antibodies and Fragments. An asthma related Ig derived protein of the invention can be of any class (IgG, IgA, IgM, IgE, IgD, etc.) or isotype and can comprise a kappa or lambda light chain. In one embodiment, the human Ig derived protein comprises an IgG heavy chain or defined fragment, for example, at least one of isotypes, IgG1, IgG2, IgG3 or IgG4. Ig derived proteins of this type can be prepared by employing a transgenic mouse or other trangenic non-human mammal comprising at least one human light chain (e.g., IgG, IgA and IgM (e.g., γ1, γ2, γ3, γ4) transgenes as described herein and/or as known in the art. In another embodiment, the anti-human asthma related Ig derived protein thereof comprises an IgG1 heavy chain and a IgG1 light chain.

At least one Ig derived protein of the invention binds at least one specified epitope specific to at least one asthma related protein, subunit, fragment, portion or any combination thereof. The at least one epitope can comprise at least one Ig derived protein binding region that comprises at least one portion of said protein, which epitope is preferably comprised of at least one extracellular, soluble, hydrophillic, external or cytoplasmic portion of said protein. As non-limiting examples, (a) an asthma related Ig derived protein specifically binds at least one epitope comprising at least 1-3, to the entire amino acid sequence, selected from the group consisting of at least one subunit of human IL-13. The at least one specified epitope can comprise any combination of at least one amino acid of human interleukin-13, e.g., at least one of 1-10, 10-20, 20-30, 30-40, 40-50, 50-60, 60-70, 70-80, 80-90, 90-100, 100-110, 110-120, 120-130, 130-140, and/or 14-145 of SEQ ID NO:1.

Generally, the human Ig derived protein or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. As a non-limiting example, the Ig derived protein or antigen-binding portion or variant can comprise at least one of the heavy chain CDR3, and/or a light chain CDR3. In a particular embodiment, the Ig derived protein or antigen-binding fragment can have an antigen-binding region that comprises at least a portion of at least one heavy chain CDR (i.e., CDR1, CDR2 and/or CDR3) having the amino acid sequence of the corresponding CDRs 1, 2 and/or 3. In another particular embodiment, the Ig derived protein or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3. Such Ig derived proteins can be prepared by chemically joining together the various portions (e.g., CDRs, framework) of the Ig derived protein using conventional techniques, by preparing and expressing a (i.e., one or more) nucleic acid molecule that encodes the Ig derived protein using conventional techniques of recombinant DNA technology or by using any other suitable method.

The anti-human asthma related Ig derived protein can comprise at least one of a heavy or light chain variable region having a defined amino acid sequence. For example, in a preferred embodiment, the anti-human asthma related Ig derived protein comprises at least one of at least one heavy chain variable region and/or at least one light chain variable region. Human Ig derived proteins that bind to human asthma related proteins or fragments and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human asthma related proteins or fragments thereof to elicit the production of Ig derived proteins. If desired, the Ig derived protein producing cells can be isolated and hybridomas or other immortalized Ig derived protein-producing cells can be prepared as described herein and/or as known in the art. Alternatively, the Ig derived protein, specified portion or variant can be expressed using the encoding nucleic acid or portion thereof in a suitable host cell.

The invention also relates to Ig derived proteins, antigen-binding fragments, immunoglobulin chains and CDRs comprising amino acids in a sequence that is substantially the same as an amino acid sequence described herein. Preferably, such Ig derived proteins or antigen-binding fragments and Ig derived proteins comprising such chains or CDRs can bind human asthma related proteins or fragments with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes

The amino acids that make up asthma related Ig derived proteins or specified portions or variants of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc.,New York, 1994):

| SINGLE | THREE | | |
|---|---|---|---|
| LETTER | LETTER | | THREE NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

An asthma related Ig derived protein of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given asthma related polypeptide will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an asthma related Ig derived protein of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one asthma related neutralizing activity. Sites that are critical for Ig derived protein binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

The Ig derived proteins or specified portions or variants of the present invention, or specified variants thereof, can comprise any number of contiguous amino acid residues from an Ig derived protein of the present invention, wherein that number is selected from the group of integers consisting of from 10-100% of the number of contiguous residues in a(n) asthma related Ig derived protein or specified portion or variant. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

In another aspect, the invention relates to human Ig derived proteins and antigen-binding fragments, as described herein, which are modified by the covalent attachment of an organic moiety. Such modification can produce an Ig derived protein or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms, as known in the art.

The modified Ig derived proteins and antigen-binding fragments of the invention can comprise one or more organic moieties that are covalently bonded, directly or indirectly, to the Ig derived protein or specified portion or variant. Each organic moiety that is bonded to an Ig derived protein or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an Ig derived protein modified by the covalent attachment of polylysine is encompassed by the invention. Hydrophilic polymers suitable for modifying Ig derived proteins of the invention can be linear or branched and include, for example, polyalkane glycols (e.g., PEG, monomethoxy-polyethylene glycol (mPEG), PPG and the like), carbohydrates (e.g., dextran, cellulose, oligosccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the Ig derived protein of the invention has a molecular weight of about 800 to about 150,000 Daltons as a separate molecular entity. For example PEG₅₀₀₀ and PEG_{20,000}, wherein the subscript is the average molecular weight of the polymer in Daltons, can be used.

The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying Ig derived proteins of the invention can be saturated or can contain one or more units of unsaturation. Fatty acids that are suitable for modifying Ig derived proteins of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoa (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human Ig derived proteins and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. A(n) “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. A(n) aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). A(n) activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified Ig derived proteins of the invention can be produced by reacting a human Ig derived protein or antigen-binding fragment with a modifying agent. For example, the organic moieties can be bonded to the Ig derived protein in a non-site specific manner by employing an amine-reactive modifying agent, for example, an NHS ester of PEG. Modified human Ig derived proteins or antigen-binding fragments can also be prepared by reducing disulfide bonds (e.g., intra-chain disulfide bonds) of an Ig derived protein or antigen-binding fragment. The reduced Ig derived protein or antigen-binding fragment can then be reacted with a thiol-reactive modifying agent to produce the modified Ig derived protein of the invention. Modified human Ig derived proteins and antigen-binding fragments comprising an organic moiety that is bonded to specific sites of an Ig derived protein of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

Asthma Related Ig Derived Protein Compositions

The present invention also provides at least one asthma related Ig derived protein composition comprising at least one, at least two, at least three, at least four, at least five, at least six or more asthma related Ig derived proteins or specified portions or variants thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the asthma related Ig derived protein amino acid sequence, or specified fragments, domains or variants thereof. Such composition percentages can be by at least one of weight, volume, concentration, molarity, or molality, or any combination therof, as liquid or dry solutions, mixtures, suspension, emulsions or colloids, as known in the art or as described herein.

Asthma related Ig derived proteins compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Remington: The Science & Practice of Pharmacy”, 19^{th} ed., Williams & Williams, (1995). Pharmaceutically acceptable carriers can be routinely selected that are suitable for the mode of administration, solubility and/or stability of the asthma related composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/Ig derived protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Asthma related Ig derived protein compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, asthma related Ig derived protein compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the asthma related compositions according to the invention are known in the art, e.g., as listed in Remington: The Science & Practice of Pharmacy, 19^{th} ed., Williams & Williams, (1995), and in the Physician's Desk Reference, 52^{nd} ed., Medical Economics, Montvale, N.J. (1998), the disclosures of which are entirely incorporated herein by reference. Preferrred carrier or excipient materials are carbohydrates (e.g., saccharides and alditols) and buffers (e.g., citrate) or polymeric agents.

Such asthma related compositions of the invention can optionally further comprise at least one selected from an asthma-related therapeutic, a TNF antagonist (e.g., but not limited to a TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonistm. Suitable amounts and dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Formulations

As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one asthma related Ig derived protein in a pharmaceutically acceptable formulation.

Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

The invention further comprises an article of manufacture, comprising packaging material, a first vial comprising lyophilized at least one asthma related Ig derived protein or specified portion or variant, and a second vial comprising an aqueous diluent of prescribed buffer or preservative, wherein said packaging material comprises a label that instructs a patient to reconstitute the at least one asthma related Ig derived protein in the aqueous diluent to form a solution that can be held over a period of twenty-four hours or greater.

The range of at least one asthma related Ig derived protein in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Other excipients, e.g. isotonicity agents, buffers, antioxidants, preservative enhancers, can be optionally and preferably added to the diluent. A(n) isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

To prepare a suitable formulation, for example, a measured amount of at least one asthma related Ig derived protein in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed formulations can be provided to patients as clear solutions or as dual vials comprising a vial of lyophilized at least one asthma related Ig derived protein that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The solutions of at least one asthma related Ig derived protein in the invention can be prepared by a process that comprises mixing at least one Ig derived protein in an aqueous diluent.

Mixing is carried out using conventional dissolution and mixing procedures. To prepare a suitable diluent, for example, a measured amount of at least one Ig derived protein in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

Recognized devices comprising these single vial systems include those pen-injector devices for delivery of a solution such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one asthma related Ig derived protein in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one asthma related Ig derived protein and a selected buffer, preferably a phosphate buffer containing saline or a chosen salt. Mixing the at least one Ig derived protein and buffer in an aqueous diluent is carried out using conventional dissolution and mixing procedures. To prepare a suitable formulation, for example, a measured amount of at least one Ig derived protein in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one asthma related Ig derived protein that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one asthma related Ig derived protein in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating asthma related conditions, in a cell, tissue, organ, animal, or patient including, but not limited to, at least one of asthma, bronchial inflammation, excess bronchial mucus or plugs, lung tissue damage, eosinophil accumulation, bronchospasm, narrowing of breathing airways, airway hypersensitivity, airway remodeling, associated pulmonary or sinus inflammation leading to at least one of inspatory or expiatory airway, wheezing, breathlessness, chest tightness, coughing, dyspnea, burning, airway edema, excess mucus, bronchospasm, tachypnea, tachycardia, cyanosis, allergic rhinitis, infections (e.g., fungal or bacterial), allergy; atopic dermatitis; biorhythm abnormalities; Churg-Strauss syndrome; flu vaccination; gastroesophageal reflux disease; hay fever; indoor allergies, and the like. Such a method can optionally comprise administering an effective amount of at least one composition or pharmaceutical composition comprising at least one asthma related Ig derived protein to a cell, tissue, organ, animal or patient in need of such modulation, treatment or therapy.

The present invention also provides a method for modulating or treating at least one asthma associated immune related disease, in a cell, tissue, organ, animal, or patient including, but not limited to, at least one of asthma, associated pulmonary or sinus inflammation leading to at least one of inspatory or expatory wheezing, breathlessness, chest tightness, coughing, dyspnea, burning, airway edema, excess mucus, bronchospasm, tachypnea, tachycardia, cyanosis, allergic rhinitis, infections (e.g., fungal or bacterial), and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2001), each entirely incorporated by reference.

Any method of the present invention can comprise administering an effective amount of a composition or pharmaceutical composition comprising at least one asthma related Ig derived protein to a cell, tissue, organ, animal or patient in need of such modulation, treatment or therapy. Such a method can optionally further comprise co-administration or combination therapy for treating such asthma related diseases, wherein the administering of said at least one asthma related Ig derived protein, further comprises administering, before concurrently, and/or after, at least one selected from an asthma-related therapeutic, a TNF antagonist (e.g., but not limited to a TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an anti parasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, an asthma related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, domase alpha (Pulmozyme), a cytokine or a cytokine antagonistm. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Asthma Related Therapies

Asthma related therapies that can optionally be combined with at least one asthma related Ig derived protein of the present invention for methods or compositions of the present invention, include any medication or treatment that can be used to treat an asthma related condition, disease, symptom or the like. Specific non-limiting examples of asthma therapies that are optionally included in methods of the present invention include, beta-2 agonists, anticholinergics, corticosteroids, glucocorticosteroids, anti-allergenics, anti-inflammatories, bronchiodialators, expectorants, allergy medications, cromolyn sodium, albuterol, Ventolin™, Proventil™; beclomethasone dipropionate inhaler, Vanceril™; budesonide inhaler, Pulmicort Turbuhaler™, Pulmicort Respules™; fluticasone and salmeterol oral inhaler, Advair™ Diskus; fluticasone propionate oral inhaler, Flovent™; hydrocortisone oral, Hydrocortone™, Cortef™; ipratropium bromide inhaler, Atrovent™; montelukast, Singulair™; prednisone, Deltasone™, Liquid Pred™; salmeterol, Serevent™; terbutaline, Brethine™; Bricanyl™; theophylline, Theo-Dur™, Respbid™, Slo-Bid™, Theo-24™, Theolair™, Uniphyl™, Slo-Phyllin™; triamcinolone acetonide inhaler, Azmacort™; methotrexate (MTX); interleukin antagonists such as IL-4, IL-5, IL-12 antibodies, receptor proteins or antagonists, and antagonist fusion proteins, IgE antibodies and antagonists, CD4 antagonists, antileukotrienes, platlet activating factor, thromoboxane antagonists, tryptase inhibitors, NK2 receptor antagonists, ipratropium, thephyllene, disodium chromoglycate (DSCG), functional or structural analogs thereof, and derivatives or variants thereof, and the like.

Historically, one of the first medications used for asthma was adrenaline (epinephrine). Adrenaline has a rapid onset of action in opening the airways (bronchodilation). It is still often used in emergency situations for asthma. Unfortunately, adrenaline has many side- effects including rapid heart rate, headache, nausea, vomiting, restlessness, and a sense of panic.

Medications chemically similar to adrenaline have been developed. These medications, called beta-2 agonists, have the bronchodilating benefits of adrenaline without many of its unwanted side-effects. Beta-2 agonists are inhaled bronchodilators which are called “agonists” because they promote the action of the beta-2 receptor of bronchial wall muscle. This receptor acts to relax the muscular wall of the airways (bronchi), resulting in bronchodilation. The bronchodilator action of beta-2 agonists starts within minutes after inhalation and lasts for about 4 hours. Examples of these medications include albuterol (Ventolin, Proventil), metaproterenol (Alupent), pirbuterol acetate (Maxair), and terbutaline sulfate (Brethaire).

A new group of long-acting beta-2 agonists has been developed with a sustained duration of effect of twelve hours. These inhalers can be taken twice a day. Salmeterol xinafoate (Serevent) is an example of this group of medications. The long-acting beta-2 agonists are generally not used for acute attacks. Beta-2 agonists can have side effects, such as anxiety, tremor, palpitations or fast heart rate, and lowering of blood potassium.

Just as beta-2 agonists can dilate the airways, beta blocker medications impair the relaxation of bronchial muscle by beta-2 receptors and can cause constriction of airways, aggravating asthma. Therefore, beta blockers, such as the blood pressure medications propanolol (Inderal), and atenolol (Tenormin), should be avoided by asthma patients.

The anticholinergic agents act on a different type of nerves than the beta-2 agonists to achieve a similar relaxation and opening of the airway passages. These two groups of bronchodilator inhalers when used together can produce an enhanced bronchodilation effect. An example of a commonly used anticholinergic agent is ipratropium bromide (Atrovent). Ipratropium takes longer to work as compared with the beta-2 agonists, with peak effectiveness occurring two hours after intake and lasting six hours. Anticholinergic agents can also be very helpful medications for patients with emphysema.

When symptoms of asthma are difficult to control with beta-2 agonists, inhaled corticosteroids (cortisone) are often added. Corticosteroids can improve lung function and reduce airway obstruction over time. Examples of inhaled corticosteroids include beclomethasone dipropionate (Beclovent, Beconase, Vancenase, and Vanceril), triamcinolone acetonide (Azmacort), and flunisolide (Aerobid). The ideal dose of corticosteroids is still unknown. The side-effects of inhaled corticosteroids include hoarseness, loss of voice, and oral yeast infections. Early use of inhaled corticosteroids may prevent irreversible damage to the airways.

Cromolyn sodium (Intal) prevents the release of certain chemicals in the lungs, such as histamine, which can cause asthma. Exactly how cromolyn works to prevent asthma needs further research. Cromolyn is not a corticosteroid and is usually not associated with significant side effects. Cromolyn is useful in preventing asthma but has limited effectiveness once acute asthma starts. Cromolyn can help prevent asthma triggered by exercise, cold air, and allergic substances, such as cat dander. Cromolyn may be used in children as well as adults.

Theophylline (Theodur, Theoair, Slo-bid, Uniphyl, Theo-24) and aminophylline are examples of methylxanthines. Methylxanthines are administered orally or intravenously. Before the inhalers became popular, methylxanthines were the mainstay of treatment of asthma. Caffeine that is in common coffee and soft drinks is also a methylxanthine drug! Theophylline relaxes the muscles surrounding the air passages, and prevents certain cells lining the bronchi (mast cells) from releasing chemicals, such as histamine, which can cause asthma. Theophylline can also act as a mild diuretic, causing an increase in urination. For asthma that is difficult to control, methylxanthines can still play an important role. Dosage levels of theophylline or aminophylline are closely monitored. Excessive levels can lead to nausea, vomiting, heart rhythm problems, and even seizures. In certain medical conditions, such as heart failure or cirrhosis, dosages of methylxanthines are lowered to avoid excessive blood levels. Drug interactions with other medications, such as cimetidine (Tagamet), calcium channel blockers (Procardia), quinolones (Cipro), and allopurinol (Xyloprim) can further affect drug blood levels.

Corticosteroids are given orally for severe asthma unresponsive to other medications. Unfortunately, high doses of corticosteroids over long periods can have serious side effects, including osteoporosis, bone fractures, diabetes mellitus, high blood pressure, thinning of the skin and easy bruising, insomnia, emotional changes, and weight gain.

Expectorants help thin airway mucus, making it easier to clear the mucus by coughing. Potassium iodide is commonly used but has the potential side-effects of acne, increased salivation, hives, and thyroid problems. Guaifenesin (Entex, Humibid) can increase the production of fluid in the lungs and help thin the mucus, but can also be an airway irritant for some people.

In addition to bronchodilator medications for those patients with atopic asthma, avoiding allergens or other irritants can be very important. In patients who cannot avoid the allergens, or in those whose symptoms cannot be controlled by medications, allergy shots are considered. The benefits of allergy shots (desensitization) in the prevention of asthma has not been firmly established. Some doctors are still concerned about the risk of anaphylaxis, which occurs in 1 in 2 million doses given. Allergy shots most commonly benefit children allergic to house dust mites. Other benefits can be seen with grass pollen, ragweed, and animal dander

In some asthma patients, avoidance of aspirin, or other NSAIDs (commonly used in treating arthritis inflammation) is important. In other patients, adequate treatment of backflow of stomach acid (esophageal reflux) prevents irritation of the airways. Measures to prevent esophageal reflux include medications, weight loss, dietary changes, and stopping cigarettes, coffee, and alcohol. Examples of medications used to reduce reflux include omeprazole (Prilosec), and ranitidine (Zantac). Patients with severe reflux problems causing lung problems may need surgery to strengthen the esophageal sphincter in order to prevent acid reflux (fundoplication surgery).

TNF Antagonists

TNF antagonists suitable for compositions, combination therapy, co-administration, devices and/or methods of the present invention (further comprising at least one anti body, specified portion and variant thereof, of the present invention), include, but are not limited to, anti-TNF Ig derived proteins, antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor Ig derived protein,” “TNF Ig derived protein,” “TNFα Ig derived protein,” or fragment and the like decreases, blocks, inhibits, abrogates or interferes with TNFα activity in vitro, in situ and/or preferably in vivo. For example, a suitable TNF human Ig derived protein of the present invention can bind TNFα and includes anti-TNF Ig derived proteins, antigen-binding fragments thereof, and specified mutants or domains thereof that bind specifically to TNFα. A suitable TNF anttibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric Ig derived protein cA2 consists of the antigen binding variable region of the high-affinity neutralizing mouse anti-human TNFα IgG1 Ig derived protein, designated A2, and the constant regions of a human IgG1, kappa immunoglobulin. The human IgG1 Fc region improves allogeneic Ig derived protein effector function, increases the circulating serum half-life and decreases the immunogenicity of the Ig derived protein. The avidity and epitope specificity of the chimeric Ig derived protein cA2 is derived from the variable region of the murine Ig derived protein A2. In a particular embodiment, a preferred source for nucleic acids encoding the variable region of the murine Ig derived protein A2 is the A2 hybridoma cell line.

Chimeric A2 (cA2) neutralizes the cytotoxic effect of both natural and recombinant human TNFα in a dose dependent manner. From binding assays of chimeric Ig derived protein cA2 and recombinant human TNFα, the affinity constant of chimeric Ig derived protein cA2 was calculated to be 1.04×10¹⁰M⁻¹. Preferred methods for determining monoclonal Ig derived protein specificity and affinity by competitive inhibition can be found in Harlow, et al., Ig derived proteins: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2004); Kozbor et al., Immunol. Today, 4:72-79 (1983); Ausubel et al., eds. Current Protocols in Molecular Biology, Wiley Interscience, New York (1987-2004); and Muller, Meth. Enzymol., 92:589-601 (1983), which references are entirely incorporated herein by reference.

In a particular embodiment, murine monoclonal Ig derived protein A2 is produced by a cell line designated c134A. Chimeric Ig derived protein cA2 is produced by a cell line designated cl68A.

Additional examples of monoclonal anti-TNF Ig derived proteins that can be used in the present invention are described in the art (see, e.g., U.S. Pat. No. 5,231,024; Möller, A. et al., Cytokine 2(3):162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules. Preferred TNF receptor molecules useful in the present invention are those that bind TNFa with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention.

Truncated forms of the TNF receptors, comprising the ECD, have been detected in urine and serum as 30 kDa and 40 kDa TNFα inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. A(n) example of such a TNF immunoreceptor fusion molecule is TNF receptor/lgG fusion protein. TNF immunoreceptor fusion molecules and methods for their production have been described in the art (Lesslauer et al., Eur. J. Immunol. 21:2883-2886 (1991); Ashkenazi etal., Proc. Natl. Acad. Sci. USA 88:10535-10539 (1991); Peppel etal., J. Exp. Med. 174:1483-1489 (1991); Kolls etal., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNFα with high affinity and possess low immunogenicity). A functional equivalent of TNF receptor molecule also includes modified TNF receptor molecules that functionally resemble TNF receptor molecules that can be used in the present invention (e.g., bind TNFα with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2004).

Cytokines include any known cytokine. See, e.g., CopewithCytokines.com. Cytokine antagonists include, but are not limited to, any Ig derived protein, fragment or mimetic, any soluble receptor, fragment or mimetic, any small molecule antagonist, or any combination thereof.

Therapeutic Treatments. Any method of the present invention can comprise a method for treating an asthma related mediated disorder, comprising administering an effective amount of a composition or pharmaceutical composition comprising at least one asthma related Ig derived protein to a cell, tissue, organ, animal or patient in need of such modulation, treatment or therapy.

Typically, treatment of pathologic conditions is effected by administering an effective amount or dosage of at least one asthma related Ig related protein composition that total, on average, a range from at least about 0.01 to 500 milligrams of at least one asthma relatedlg derived protein/kilogram of patient per dose, and preferably from at least about 0.1 to 100 milligrams Ig derived protein/kilogram of patient per single or multiple administration, depending upon the specific activity of contained in the composition. Alternatively, the effective serum concentration can comprise 0.1-5000 μg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one Ig derived protein of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the Ig derived protein can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Alternative Administration

Many known and developed modes of can be used according to the present invention for administering pharmaceutically effective amounts of at least one asthma related Ig derived protein according to the present invention. While pulmonary administration is used in the following description, other modes of administration can be used according to the present invention with suitable results.

asthma related Ig derived proteins of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aquous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of at least one asthma related Ig derived protein by parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal means. Protein, Ig derived protein compositions can be prepared for use for parenteral (e.g., but not limited to, subcutaneous, intramuscular or intravenous) administration particularly in the form of liquid solutions or suspensions; for use in vaginal or rectal administration particularly in semisolid forms such as creams and suppositories; for buccal, or sublingual administration particularly in the form of tablets or capsules; or intranasally particularly in the form of powders, nasal drops or aerosols or certain agents; or transdermally particularly in the form of a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration

For pulmonary administration, preferably at least one asthma related Ig derived protein composition is delivered in a particle size effective for reaching the lower airways of the lung or sinuses. According to the invention, at least one asthma related Ig derived protein can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of Ig derived protein or specified portion or variants are also known in the art. All such devices can use of formulations suitable for the administration for the dispensing of Ig derived protein in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one asthma related Ig derived protein is delivered by a dry powder inhaler or a sprayer. There are a several desirable features of an inhalation device for administering at least one Ig derived protein of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of Asthma Related Ig Derived Protein Compositions as a Spray

A spray including asthma related Ig derived protein composition protein can be produced by forcing a suspension or solution of at least one Asthma related Ig derived protein through a nozzle under pressure. The nozzle size and configuration, the applied pressure, and the liquid feed rate can be chosen to achieve the desired output and particle size. A(n) electrospray can be produced, for example, by an electric field in connection with a capillary or nozzle feed. Advantageously, particles of at least one asthma related Ig derived protein composition protein delivered by a sprayer have a particle size less than about 10 μm, preferably in the range of about 1 μm to about 5 μm, and most preferably about 2 μm to about 3 μm.

Formulations of at least one asthma related Ig derived protein composition protein suitable for use with a sprayer typically include Ig derived protein composition protein in an aqueous solution at a concentration of about 0.1 mg to about 100 mg of at least one asthma related Ig derived protein composition protein per mil of solution or mg/gm, or any range or value therein, e.g., but not limited to, 0.1, 0.2., 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/ml or mg/gm. The formulation can include agents such as an excipient, a buffer, an isotonicity agent, a preservative, a surfactant, and, preferably, zinc. The formulation can also include an excipient or agent for stabilization of the Ig derived protein composition protein, such as a buffer, a reducing agent, a bulk protein, or a carbohydrate. Bulk proteins useful in formulating Ig derived protein composition proteins include albumin, protamine, or the like. Typical carbohydrates useful in formulating Ig derived protein composition proteins include sucrose, mannitol, lactose, trehalose, glucose, or the like. The Ig derived protein composition protein formulation can also include a surfactant, which can reduce or prevent surface-induced aggregation of the Ig derived protein composition protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as asthma related Ig derived proteins, or specified portions or variants, can also be included in the formulation.

Administration of Asthma Related Ig Derived Protein Compositions by a Nebulizer

Ig derived protein composition protein can be administered by a nebulizer, such as jet nebulizer or an ultrasonic nebulizer. Typically, in ajet nebulizer, a compressed air source is used to create a high-velocity air jet through an orifice. As the gas expands beyond the nozzle, a low-pressure region is created, which draws a solution of Ig derived protein composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of Ig derived protein composition protein either directly or through a coupling fluid, creating an aerosol including the Ig derived protein composition protein. Advantageously, particles of Ig derived protein composition protein delivered by a nebulizer have a particle size less than about 10 μm, preferably in the range of about 1 μm to about 5 μm, and most preferably about 2 μm to about 3 μm.

Formulations of at least one asthma related Ig derived protein suitable for use with a nebulizer, either jet or ultrasonic, typically include a concentration of about 0.1 mg to about 100 mg of at least one asthma related Ig derived protein protein per ml of solution. The formulation can include agents such as an excipient, a buffer, an isotonicity agent, a preservative, a surfactant, and, preferably, zinc. The formulation can also include an excipient or agent for stabilization of the at least one asthma related Ig derived protein composition protein, such as a buffer, a reducing agent, a bulk protein, or a carbohydrate. Bulk proteins useful in formulating at least one asthma related Ig derived protein composition proteins include albumin, protamine, or the like. Typical carbohydrates useful in formulating at least one asthma related Ig derived protein include sucrose, mannitol, lactose, trehalose, glucose, or the like. The at least one asthma related Ig derived protein formulation can also include a surfactant, which can reduce or prevent surface-induced aggregation of the at least one asthma related Ig derived protein caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as Ig derived protein protein can also be included in the formulation.

Administration of Asthma Related Ig Derived Protein Compositions By A Metered Dose Inhaler

In a metered dose inhaler (MDI), a propellant, at least one asthma related Ig derived protein or specified portion or variant, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of Ig derived protein composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one asthma related Ig derived protein for use with a metered-dose inhaler device will generally include a finely divided powder containing at least one asthma related Ig derived protein as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one asthma related Ig derived protein as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one asthma related Ig derived protein compositions via devices not described herein.

Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 are used to deliver biologically active agents orally are known in the art.

Mucosal Formulations and Administration

For absorption through mucosal surfaces, compositions and methods of administering at least one asthma related Ig derived protein include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. Nos. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. No. 5,849,695).

Transdermal Formulations and Administration

For transdermal administration, the at least one asthma related Ig derived protein is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. No. 5,814,599).

Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. No. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### EXAMPLE 1

### Cloning and Expression of Asthma Related Immunoglobulin Protein in Mammalian Cells

A typical mammalian expression vector contains at least one promoter element, which mediates the initiation of transcription of MRNA, the Ig derived protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded Ig derived protein or specified portion or variant. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of Ig derived protein or specified portion or variants.

The expression vectors pC1 and pC4 contain the strong promoter (LTR) of the Rous Sarcoma Virus (Cullen, et al., Molec. Cell. Biol. 5:438447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

Cloning and Expression in CHO Cells

The vector pC4 is used for the expression of asthma related Ig derived protein or specified portion or variant. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the asthma related in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete asthma related Ig derived protein is used, corresponding to HC and LC variable regions of an asthma related Ig derived protein of the present invention, according to known method steps. Isolated nucleic acid encoding a suitable human constant region (i.e., HC and LC regions) is also used in this construct (e.g., as provided in vector p1351).

The isolated variable and constant region encoding DNA and the dephosphorylated vector are then ligated with T4 DNA ligase. E. coli HB101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from Tn5 encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100 -200 mM. Expression of the desired gene product is analyzed, for instance, by SDS-PAGE and Western blot or by reverse phase HPLC analysis.

The completely human anti- asthma related protein Ig derived proteins are further characterized. Several of generated Ig derived proteins are expected to have affinity constants between 1×10⁹ and 9×10¹². Such high affinities of these fully human monoclonal Ig derived proteins make them suitable for therapeutic applications in asthma related protein-dependent diseases, pathologies or related conditions.

### EXAMPLE 2

### Use of IL-13 Antibodies of the Present Invention in Animal Models of Asthma

Interleukin 13 (IL-13) is a pleiotropic cytokine mainly produced by Th2 cells. Over-expression of IL-13 in the lung in mice animal models of asthma with recombinant IL-13 intranasally induced airway hyperresponsiveness (AHR), mucus gland hyperplasia, eotaxin production, pulmonary eosinophila and subepithelia fibrosis. Blocking IL-13 using either the IL-13 receptor-Ig fusion protein or polyclonal antiserum in asthmatic mice has been shown to significantly inhibited AHR, mucus production, airway inflammation and fibrosis. However, the use of monoclonal antibodies to IL-13 has not been shown to affect these markers of asthma.

The following results show that IL-13 is a key player in asthma pathogenesis, and that IL-13 specific monoclonal antibody therapy is expected to provide therapeutic efficacy in humans with asthma or asthma-like conditions. To prove the concept, we have developed a rat anti-mouse IL-13 neutralizing monoclonal antibody (mAb) and tested its effects on OVA induced acute asthma responses in mice. IL-13 was up-regulated in the lung during OVA induced asthma responses. When administered at the challenge stage, the anti-IL-13 monoclonal antibody significantly inhibited AHR, goblet cell hyperplasia and mucus production. Furthermore, the antibody treatment also inhibited the production of IL-5, IL-6, eotaxin, KC, MIP-1 and MCP-1 in the lung. These results clearly demonstrated that IL-13 plays an important role in asthma responses, and suggest that a monoclonal antibody to IL-13 would be an effective therapeutic agent in the treatment of asthma.

An rat-anti-mouse IL-13 monoclonal antibody (mAb) neutralizes mouse IL-13 activity in a cell-based bioassay. B9 cells were incubated with 5 ng/ml of mouse IL-13 and different concentrations of the rat-anti-mouse IL-13 mAb for 3 days. The IL-13 dependent cell proliferation was measured using a luminescent ATP detection kit.

Testing of the anti-IL-13 effects on an acute asthma-like response. BALB/c mice were sensitized i.p. with OVA/Alum on day 1 and day 8, and challenged with OVA/PBS intranasal on day 22-24. On day 22 and day 24, 0.5 mg/mouse of the anti-mL-13 mAb was given through intravenous injections. On day 25, AHR was measured, mice were then euthanized and samples were collected.

Anti-IL-13 inhibits methacholine induced Airway hyper-responsiveness (AHR) in OVA sensitized/challenged mice. Twenty-four hours after the last OVA intranasal challenge, mice were stimulated with aerosolized PBS or increasing doses of methacholine (5, 10 and 20 mg/ml). AHR was measured using whole body plethysmography (Buxco).

Anti IL-13 mAb did not reduce cellular infiltration in the airways of OVA sensitized/challenged mice. On day 25, mice were euthanized, and their lungs were lavaged. Cells in the bronchoalveolar lavage (BAL) were collected and cytospin preparations were made. Different cell types in the BAL were analyzed by differential cell counts.

Anti-IL-13 inhibits goblet cell hyperplasia and mucus production in the OVA sensitized/challenged mice. On day 25, the mice were euthanized, BAL collected and their left lungs were fixed. The mucus producing goblet cells were visualized by periodic acid-shiff (PAS) staining.

The mucus production in all the bronchioles evaluated. The intensity of mucus production was analyzed using mucus scores.

0: no mucus-producing goblet

1: mucus-producing goblet cells cover <⅓ of the bronchial epithelium

2: mucus-producing goblet cells cover >⅓ of the bronchial epithelium

3: mucus-producing goblet cells cover most of the bronchial epithelium

There were significantly more mucus free bronchioles and significantly less bronchioles with higher mucus scores in anti-IL-13 treated mice.

Anti IL-13 mAb did not reduce the serum levels of antigen-specific immunoglobulin. On day 25, mice were euthanized, and serum collected. The OVA specific IgE, IgG1 and IgG2a levels were measured using ELISA.

Anti IL-13 mAB significantly inhibited airway IL-5 and IL-6 levels of OVA sensitized/challenged mice. On day 25, mice were euthanized, and BAL fluids (BALF) collected. IL-5 and IL-6 levels in the BALF were measured by ELISA.

Anti-IL-13 mAb inhibited a panel of chemokine production in the lung of the OVA sensitized/challenged mice. On day 25, the mice were euthanized, and their right lungs were homogenized and chemokine levels in the homogenates were measured using ELISA. The anti-IL-13 mAb significantly inhibited Eotaxin, RANTES, KC, MCP-1 and MIP-1 production in the lung without altering the levels of RANTES.

Thus, neutralizing anti-IL-13 mabs were shown to significantly suppress several aspects of asthma-like responses in an acute mouse model. Anti-IL-13 inhibited AHR, mucus production and cytokine/chemokine levels without a significant reduction of the airway cellular infiltration and serum IgE levels. These data may suggest that mucus production, AHR and pulmonary inflammation may be regulated by different mechanisms. Our results also indicate that a mAb to IL-13 would be expected to be an effective therapeutic agent in the treatment of asthma in mammals and in humans.

### EXAMPLE 3

### Anti-IL-13 Monoclonal Antibody Inhibits Airway Hyperresponsiveness, Inflammation and Airway Remodeling

Abstract: Asthma is a chronic inflammatory disease characterized by reversible bronchial constriction, pulmonary inflammation and airway remodeling. Current standard therapies for asthma have provided symptomatic control but failed to target the underlying disease pathology. Furthermore, no therapeutic agent is effective in preventing airway remodeling, which includes epithelial damage, mucus gland hyperplasia, airway smooth muscle hypertrophy, and sub-epithelial fibrosis. Interleukin 13 (IL-13) is a pleiotropic cytokine produced mainly by T cells. A substantial amount of evidence suggests that IL-13 plays a critical role in the pathogenesis of asthma. Therefore, a neutralizing anti-IL-13 monoclonal antibody could provide therapeutic benefits to asthmatic patients. To prove the concept we have generated a neutralizing rat anti-mouse IL-13 monoclonal antibody, and tested its effects in a chronic mouse model of asthma. Chronic asthma-like response was induced in OVA sensitized mice by repeated intranasal OVA challenges. After weeks of challenge, mice developed airway hyperresponsiveness (AHR) to mechacholine stimulation, severe airway inflammation, hyper mucus production, and subepithelial fibrosis. When given at the time of each intranasal OVA challenge, anti-IL-13 antibody significantly suppressed AHR, eosinophil infiltration, pro-inflammatory cytokine/chemokine production, serum IgE, and most interestingly airway remodeling. Taken together, these results strongly suggested that a neutralizing anti-human IL-13 monoclonal antibody could be an effective therapeutic agent for asthma.

Introduction:

Asthma is a chronic disease characterised by reversible airway obstruction, pulmonary inflammation and airway remodelling [3-1]. Although current standard therapies (including corticosteroid and β2 receptor agonist) are effective in providing symptomatic control for the majority of asthma patients, patients with severe or difficult asthma do not response to these therapies well [3-2]. Furthermore, no therapeutic agent is effective in preventing airway remodelling, which involves epithelial damage, mucus gland hyperplasia, airway smooth muscle hypertrophy, and sub-epithelial fibrosis [3-3]. These structural changes of the airway, caused by chronic airway inflammation and a repeated damage/repair process, is a fundamental component for the development of irreversible airway hyperresponsiveness and directly contributes to the severity of asthma [3-4]. Therefore, developing the therapeutic agents that can suppress airway tissue remodelling would fulfil the unmet medical need in the treatment of asthma.

IL-13 is an immunomodulating cytokine produced by activated T cells and many other cell types [3-5]. The involvement of IL-13 in asthma pathogenesis has been extensively studied [3-6]. In humans, IL-13 levels are upregulated in asthmatics both systemically and in the lung during asthmatic attacks [3-7 to 3-12]. Certain IL-13 polymorphosims are associated with high IgE levels and high risk for asthma development [3-13]. Furthermore, IL-13 exhibits stimulatory activities to multiple cell types that are involved in asthma, including B cells, mast cells, eosinophils, pulmonary epithelial cells, fibroblasts and airway smooth muscle cells [3-5, 6]. In animal studies, direct administration of recombinant IL-13, or genetic over expression of IL-13 in the lung induced asthma-like airway inflammation and airway remodelling [3-14, 3-15]. Blockade of IL-13 using a receptor fusion protein inhibited allergic asthma response in mice [3-16, 3-17]. Taken together, these results strongly suggest that IL-13 plays a crucial role in the pathogenesis of asthma. Therefore, a neutralizing monoclonal antibody (mAb) to IL-13 could provide therapeutic benefits to asthmatic patients.

A neutralizing rat anti-mouse IL-13 Ab was generated using standard hybridoma 15 technology. To fully evaluate the impact of an anti-IL-13 Ab on different pathological aspects of asthma, especially on airway remodelling, a chronic asthma model was developed by repeated intranasal antigen (Ag) challenge to sensitized mice. After 5 weeks of weekly challenge, the mice developed multiple pathological features that represented those of human asthma, including airway hyperresponsiveness (AHR), severe pulmonary inflammation and airway remodelling. When administered at the same time of each Ag challenge, anti-IL-13 antibody effectively suppressed AHR, eosinophil infiltration, goblet cell hyperplasia, excessive mucus production, as well as sub-epithelial fibrosis. These results indicated that a neutralizing mAb to human IL-13 could potentially control asthma symptoms as well as preserve normal airway structure and function.

Materials and Methods:

Mice

Naïve BALB/C mice were purchased from Charles Rivers Laboratories (Raleigh N.C.). All mice were maintained under specific pathogen free conditions under a protocol approved by the Institutional Animal Care and Use Committee of Centocor.

Reagents

Ovalbumin (OVA) and methacholine were purchased from Sigma (St. Louis, Mo.).

Normal rat IgG and Peroxidase-conjugated Streptavidin were from Jackson ImmunoResearch (West Grove, Pa.). ELISA reagents for measuring serum immunoglobulin isotypes were from PharMingen (Franklin lakes, N.J.). Cytokine and chemokine ELISA kits were purchased from R & D system (Minneapolis, Minn.).

Rat anti-mouse IL-13 monoclonal antibody

The rat anti-mouse IL-13 monoclonal antibody (mAb) was generated by immunizing Sprague Dawley rats with recombinant mouse IL-13 (R & D system), and the hybridoma was developed using standard hybridoma technology. The biological activity of the anti-IL-13 antibody was measured using a cell based biological assay. B9 cell (ATCC, Rockville, Md.) exhibits a dose dependent proliferation to mouse IL-13 with an EC₅₀ of 5 ng/ml, measured with a luminescent ATP detection assay kit (Packard Bioscience, Meriden, Conn.). With a concentration of 5 ng/ml IL-13, the anti-IL-13 mAb exhibited a dose dependent inhibition of B9 cell proliferation with an IC₅₀ around 100 ng/ml.

Chronic Asthma Model and Treatment

All BALB/c mice were kept on an OVA free diet, and were used at 6 weeks (wk) of age. Mice were sensitized intraperitoneally with 100 μg OVA in 100 μl of PBS mixed with 100 μl of inject Alum (Pierce, Rockford, Ill.) on day 1, and boosted the same way on day 8. One week after the boost, mice were challenged with 100 ng of OVA in 50 μl PBS or PBS alone intranasal once a week for 5 weeks. One hour before each intranasal challenge, mice received either the anti-IL-13 mAb or normal rat IgG (500 μg/200 μl/mouse/dose) intravenously. AHR was measured one day after the last intranasal challenge, mice were then enthanized and multiple endpoints were evaluated.

Airway Hyper-Responsiveness (AHR)

AHR was assessed by measuring mechacholine induced airway resistance using whole body plethysmography (Buxco, Sharon, Conn.). Conscious mice were stimulated with aerosolized PBS followed by incremental doses of aerosolized methacholine (5-20 mg/ml in PBS) for 2 minutes. Airway resistance, representing airway responsiveness to methacholine stimulation, was monitored and expressed as enhanced pause (Penh) (Hansen and Umestu, 1999).

Broncho-Alveolar Lavage (BAL) Collection and Differential Cell Count

Mice were euthanized and their lungs perfused through the right ventricles using 3-5 ml sterile PBS until it turned white. The lungs were then flushed 2 times through the trachea with 1 ml of 2% fetal calf serum (FCS) in PBS with 15 unit/ml of heparin to obtain BAL. Supernatant of the BAL was collected after centrifugation for cytokine analysis. BAL cells were washed and resuspended in 1 ml 2% FCS PBS, and cytospin was made for differential cell count.

Detection of Different Isotypes of OVA-Specific Immunoglobulin in Serum

96 wells high binding plates (Costar, Corning, N.Y.) were coated with 100 μl/well of OVA (20 μg/ml) at 4° C. over night. For the standard curve, wells were coated with 100 μl/well of anti-mouse IgE, IgG1 or IgG2a antibody (2 μg/ml) accordingly. Plates were washed, blocked with 20% FCS PBS and washed again. 100 μl/well of serum samples or standard mouse IgE, IgG1 or IgG2a (1 μg/ml) were added and incubated for one hour at room temperature (RT). Wells were washed and 1 μg/ml of biotinylated rat anti-mouse Ig isotype was added and incubated for one hour at RT. Wells were washed and 100 μl/well of peroxidase-conjugated streptavidin (0.2 μg/ml) was added. Plates were incubated for 30 minutes at RT. Color was developed by adding 100 μl of substrate and stopped by adding 50 μl/well of 2 N H₂SO₄. Absorbance was determined at 450 nm.

Detection of Cytokine/Chemokine Levels in the Lung

The right lungs were homogenized in 1 ml of PBS and the supernatant was collected following centrifugation. The levels of different chemokines in the supernatants were assessed using ELISA kits (R & D system, Minneapolis, Minn.).

Histology

The left lungs were fixed with 10% formalin and embedded in paraffin using standard methods. Tissues were sectioned and stained with hematoxylin and eosin (H&E) to analyze inflammation, Periodic Acid-Schiff (PAS) for mucus, and Masson's Trichrome or picric-sirius red (SR) for collagen fibers. All staining were performed with standard protocols. To semi-quantitatively analyze sub-epithelial fibrosis of bronchioles, all bronchioles in the lung tissue section of each mouse were photographed at 40×, and the SR stained areas were measured using Image-Pro Plus system. The index of collagen was calculated using the following formula: (SR positive area/total bronchial area)×100.

Statistical Analysis

Each point corresponds to mean±standard deviation (SD). Statistical differences were determined by one-way analysis of variance (ANOVA) and p<0.05 was considered to be statistically significant.

Results:

Neutralizing Anti-IL-13 mAb Inhibits AHR to Mmethacholine Stimulation

Increased airway responsiveness and sensitivity to nonspecific stimulation is a major pathological characteristic of human asthma. To evaluate the effect of anti-IL-13 mAb on airway constriction, whole body plethysmography was used to measure airway resistance to incremental concentrations of mechacholine. Mice that were sensitised with OVA but challenged with PBS for 5 weeks revealed a low baseline airway resistance (represented as Penh) with the Penh values slightly increased upon mechacholine stimulation. In contrast, sensitized mice that received weekly OVA challenge for 5 weeks exhibited dramatically increased response and sensitivity to methacholine stimulation, demonstrating AHR. Anti-IL-13 mAb, when administered at the time of each OVA challenge, significantly inhibited AHR to the levels of those in the PBS challenged mice. This result suggested that IL-13 played a critical role in the pathogenesis of AHR, and an anti-L-13 mAb probably would suppress AHR and its related symptoms in asthma.

Blockade of IL-13 Suppresses Pulmonary Inflammation

Asthma is a chronic pulmonary inflammatory disease characterized by airway and tissue infiltration of eosinophils and other cell types. To assess the effect of anti-IL-13 mAb on pulmonary inflammation, mice were euthanized after the last intranasal challenge, and their bronchoalveolar lavage (BAL) was collected and different types of cells counted. Mice that received 5 weeks of PBS challenge only contained a few alveolar macrophages in their BAL. In contrast, 5 weeks of OVA challenge induced massive infiltration of mononuclear cells and eosinophils, as well as some neutrophils. Anti-IL-13 treatment significantly suppressed eosinophil infiltration up to 50% without affecting the recruitment of mononuclear cells. Tissue cellular infiltration was also assessed on H&E stained lung sections. Chronic OVA challenge induced dramatic perivascular and peribronchial infiltration of various types of cells, and anti-IL-13 treatment noticeably reduced the overall intensity of the infiltration. Taken together, these results demonstrated that anti-IL-13 mAb inhibited pulmonary inflammation, especially the infiltration of eosinophils.

Inflammation associated with asthma is regulated by multiple proinflammatory mediators. To understand the potential mechanisms of anti-IL-13 mAb mediated inhibition of pulmonary inflammation, we checked the effect of anti-IL-13 mAb treatment on serum Ag specific IgE levels as well as on multiple cytokines and chemokines in the lung. Different isotypes of OVA specific immunoglobulin in the serum were measured using ELISA. Although anti-IL-13 mAb did not affect the levels of OVA specific IgG1 and IgG2a, it significantly inhibited OVA specific IgE. Cytokines/chemokines were detected either in the BAL or in the lung homogenates using ELISA. Levels of Th2 cytokines (IL-4 and IL-5) as well as proinflammation cytokines (IL-6 and TNFO) were upregulated in the chronically inflamed lungs, and anti-IL-13 mAb suppressed all these cytokines (data not shown). The production of chemokines (eotaxin, MIP-1α, and KC) were increased in the tissues homogenates of the inflamed lung, and anti-IL-13 mAb significantly inhibited the levels of all these chemokines. These data indicated that the suppression of pulmonary inflammation by anti-IL-13 mAb probably was mediated through the inhibition of multiple inflammatory mediators including Ag specific IgE, cytokines and chemokines.

Anti-IL-13 mAb Prevents Airway Remodeling

Airway remodeling represents a progressive structural change in the airways and is believed to contribute to the disease progression [3-18, 3-19]. The structural changes in the bronchial walls include goblet cell hyperplasia, mucus hypersecretion, airway smooth muscle hypertrophy, and subepithelial fibrosis. Although current standard therapies are effective in controlling asthma related symptoms, airway remodeling is persistently present in most asthma patients [3-20]. Therefore, there is a strong need for developing novel therapeutic interventions that prevent or stop the tissue remodeling process associated with asthma.

The chronic asthma model features several airway structural changes that represent those in human asthma, such as goblet cell hypertrophy, excessive mucus production and subepithelial fibrosis. Goblet cell hyperplasia and mucus hyper secretion became obvious with only two weekly OVA intranasal challenges. After 5 weeks of OVA challenge, the bronchial epithelium of almost all the small bronchioles were heavily populated with mucus containing goblet cells. Anti-IL-13 treatment totally abolished goblet cell hypertrophy and mucus production. This result further demonstrated that IL-13 was a key modulator for goblet cell differentiation and mucus induction, and anti-IL-13 mAb was effective in controlling excessive mucus production, which is one of the major causes of asthma associated fatality.

Subepithelial fibrosis represents another airway structural change that has negative impact on airway functions [3-18]. To access the potential effect of anti-IL-13 mAb on fibrosis in the chronic asthma model, tissue collagen deposition was visualized with trichrom C or Sirius red staining. Significant collagen deposition was present in the subepithelial area of most of the bronchioles from mice that received 5 weeks of OVA challenge, indicating significant subepithelial fibrosis. Anti-IL-13 mAb treatment effectively reduced the intensity of collagen deposition. To analyze supepithelial fibrosis in a more quantitative manner, sub-epithelial collagen deposition of all the bronchioles on the tissue sections of each mouse was measured using Image-Pro Plus system and presented as an index of collagen. Anti-IL-13 mAb significantly reduced the overall intensity of collagen deposition. Taken together, these results suggested that a neutralizing anti-IL-13 mAb would be effective in preventing or halting tissue fibrosis in chronic asthmatic responses.

Discussion:

IL-13 is a pleiotropic cytokine that is involved in multiple physiological and pathological processes [3-5]. To date accumulating evidence indicates that IL-13 plays a key role in the pathogenesis of asthma [3-6]. It has been demonstrated that IL-13 expression was increased in lungs of asthmatics during active asthma responses [3-7 to 3-12], and IL-13 exhibits a broad spectrum of pathological activities on many inflammatory infiltrating cells (B cells, mast cells, and eosinophil cells) as well as parenchyma cells (airway smooth muscle cells, pulmonary epithelial cells, endothelial cells and fibroblast) [3-5]. All this information strongly suggests a key involvement of IL-13 in human asthma, which makes it a very attractive target for the treatment of asthma. In this study, using a mouse model of chronic asthma, we tested the effects of an anti-IL-13 mAb on different pathological features of asthma. We found that the anti-IL-13 mAb significantly inhibited methacholine induced AHR, pulmonary inflammation, multiple cytokine/chemokine production, goblet cell hyperplasia, and subepithelial fibrosis. These results further supported a broad role IL-13 played in different aspects of asthma, and indicated the potential therapeutic benefits of an anti-IL-13 mAb in the treatment of human asthma.

Exogenous administered or transgenicaly expressed IL-13 induced AHR in mice [3-14, 3-15] and IL-13Ra2-Ig fusion protein inhibited AHR in mouse models of asthma [3-16, 3-17]. These observations strongly indicated that IL-13 was crucial in the development of AHR in asthma. However, several studies using IL-13 deficient mice demonstrated persistent AHR associated with asthma responses [3-21, 3-22, 3-23]. Although the different findings could be due to the different mouse strains and model systems used, it may also suggest that IL-13's involvement in AHR might depend on the stage and intensity of the diseases. In our study system using OVA sensitized and challenged mice, the anti-IL-13 mAb significantly inhibited methacholine induced AHR in both acute [3-24] and chronic asthma responses, which again supported the hypothesis that IL-13 played an important role in AHR. Furthermore, human airway smooth muscle cells (ASMC) directly responded to IL-13 [3-25, 3-26], and IL-13 increased contractile responses and calcium signals of ASMC to agonist stimulation [3-27]. Taken together, IL-13 may have both direct and indirect pathological impact on airway smooth muscle cells to cause AHR in asthma, and an anti-IL-13 mAb could potential reduce airway symptoms associated with AHR.

IL-13 contributes to asthma associated pulmonary inflammation probably through multiple mechanisms. IL-13 enhances IgE production [3-28] and thereby could augment the early phase inflammation. IL-13 is the major Th2 cytokine that induces the chemoattractants for eosinophils (eotaxin) [3-29] and Th2 cells (TARC and eotaxin). In the chronic asthma model, anti-IL-13 mAb dramatically reduced the levels of eotaxin as well as KC and MIP-1□, further confirming its important role in regulating chemokine production. However, in agreement with previous reports [3-16, 3-24], neutralization of IL-13 alone did not totally suppress pulmonary inflammation. Anti-IL-13 mAb only suppressed eosinophil infiltration into the airways up to 50% without affecting other cell types, and the tissue infiltration was significant albeit reduced overall intensity with the anti-IL-13 treatment. These results indicated the pathological redundancy among multiple pro-inflammatory cytokines/chemokines during an active asthma inflammation.

Human asthma is a chronic inflammatory disorder with significant airway structural changes or airway remodeling [3-19]. Airway remodeling, such as goblet cell hyperplasia, excessive mucus production and basement membrane fibrosis, probably accounts for the incomplete therapeutic efficacy of current therapeutics and continuous disease progression [3-18, 3-20]. The majority of the mouse asthma models are acute allergic pulmonary inflammatory responses with AHR. Although these models represent the phenotypes of acute asthma attacks or exacerbations, the chronic nature of human asthma and airway remodeling are not well reflected in these models. Chronic asthma models particularly addressing airway remodeling would be more relevant for novel therapy discovery. In this study, with repeated Ag intranasal challenges for 5 weeks, the sensitized mice developed significant mucus metaplasia, basement membrane thickness and supepithelial fibrosis, which provided us with valuable tools to test the role of IL-13 in airway remodeling. Other chronic asthma models, with either low dose OVA chronic aerosolization to sensitized mice or with Aspergillus fumigatus Ags, also suggested the pathogenic role of IL-13 in airway remodeling [3-30, 3-31].

Mucus metaplasia and mucus plugging are well-documented features of chronic asthma and are the major causes of asthma associated fatality [3-32, 3-33]. Accumulating evidence has demonstrated IL-13 to be the key regulator of goblet cell differentiation [3-34] and mucus hypersecretion [3-15]. In this study, despite the existence of chronic pulmonary inflammation, anti-IL-13 mAb almost totally abolished goblet cell hyperplasia and the excessive mucus secretion. Furthermore, when given at the stage of established goblet cell hypertrophy (after two weekly OVA challenges), the anti-IL-13 mAb stopped and even reversed goblet cell hypertrophy and mucus hypersecretion (G. Yang unpublished results). These results suggested that a therapeutic anti-IL-13 mAb probably could prevent the consequences related to mucus metaplasia during fatal asthma attacks.

Subepithelial fibrosis represents another major airway structural change that is associated with disease severity and correlated with a decline in pulmonary functions in human asthma [3-18, 3-35]. IL-13 transgenic expression in the lung induced extensive subepithelial fibrosis that is mediated through the over activation of TGFβ[3-36]. Collagen deposition and subepithelial fibrosis were reduced in chronic asthma models using either IL-13 deficient mice or polyclonal anti-IL-13 antibodies [3-30, 31]. Our study demonstrated that an anti-IL-13 mAb significantly suppressed collagen deposition and reduced epithelial fibrosis. Furthermore, IL-13 directly acts on human fibroblasts to enhance activation [3-37, 3-38], proliferation [3-39], collagen generation [3-40] and differentiation [3-41], which indicated a pathogenic role of IL-13 in airway fibrosis in human asthma. Therefore, a neutralizing anti-IL-13 mAb could potentially prevent and halt the tissue fibrosis in asthma and to potentially halt disease progression.

Current therapies (bronchial dilators and corticosteroids) are effective in symptomatic control of the majority of asthmatic patient, however, a small percentage of patient still present persistent symptoms despite high dose of inhaled or oral corticosteroids [3-2, 3-4]. Although the pathology of the so-called severe asthma or steroid insensitive asthma is poorly understood, IL-13 could contribute to steroid insensitivity either directly or indirectly. IL-13 levels were increased in severe asthmatics [3-10]. Higher levels of IL-13 were produced in macrophages of nocturnal asthma patients with decreased steroid responsiveness at night, and neutralizing antibodies to IL-13 reduced the decoy glucocorticoid receptor (GR) GR□ expression by macrophages in those patients [3-42]. IL-13 decreased monocyte GR binding affinity, thereby contributed to impaired GR responsiveness [3-43]. Furthermore, IL-13 enhanced the proliferating effect of steroids on lung fibroblasts [3-45], [3-46], suggesting enhanced airway remodeling. In animal models, IL-13 induced AHR and mucus production were steroid insensitive [3-44]. Taken together, these results indicated a pathogenic role of IL-13 in severe steroid insensitive asthma, and an anti-IL-13 mab may provide therapeutic benefit to this patient population.

### EXAMPLE 3 References

3-1. Hansel, T. T. and P. J. Barnes, Novel drugs for treating asthma. Curr Allergy Asthma Rep, 2001. 1(2): p. 164-73.

3-2. Garcia, G., M. Adler, and M. Humbert, Difficult asthma. Allergy, 2003. 58(2): p. 114-21.

3-3. Holgate, S. T., et al., Roles of cysteinyl leukotrienes in airway inflammation, smooth muscle function, and remodeling. J Allergy Clin Immunol, 2003. 111(1 Suppl): p. S18-34; discussion S34-6.

3-4. Wenzel, S., Pathology of difficult asthma. Paediatr Respir Rev, 2003. 4(4): p. 306-11.

3-5. Wynn, T. A., IL-13 effector functions. Annu Rev Immunol, 2003. 21: p. 425-56.

3-6. Wills-Karp, M. and M. Chiaramonte, Interleukin-13 in asthma. Curr Opin Pulm Med, 2003. 9(1): p. 21-7.

3-7. Prieto, J., et al., Increased interleukin-13 mRNA expression in bronchoalveolar lavage cells of atopic patients with mild asthma after repeated low-dose allergen provocations. Respir Med, 2000. 94(8): p. 806-14.

3-8. Lee, Y. C., et al., Serum levels of interleukins (IL)-4, IL-5, IL-13, and interferon-gamma in acute asthma. J Asthma, 2001. 38(8): p. 665-71.

3-9. Wong, C. K., et al., Proinflammatory cytokines (IL-17, IL-6, IL-18 and IL-12) and Th cytokines (IFN-gamma, IL-4, IL-10 and IL-13) in patients with allergic asthma. Clin Exp Immunol, 2001. 125(2): p. 177-83.

3-10. Simon, H. U., et al., Clinical and immunological effects of low-dose IFN-alpha treatment in patients with corticosteroid-resistant asthma. Allergy, 2003. 58(12): p. 1250-5.

3-11. Kotsimbos, T. C., P. Ernst, and Q. A. Hamid, Interleukin-13 and interleukin-4 are coexpressed in atopic asthma. Proc Assoc Am Physicians, 1996. 108(5): p. 368-73.

3-12. Bodey, K. J., et al., Cytokine profiles of BAL T cells and T-cell clones obtained from human asthmatic airways after local allergen challenge. Allergy, 1999. 54(10): p. 1083-93.

3-13. Vercelli, D., Genetics of IL-13 and functional relevance of IL-13 variants. Curr Opin Allergy Clin Immunol, 2002. 2(5): p. 389-93.

3-14. Yang, M., et al., Interleukin-13 mediates airways hyperreactivity through the IL-4 receptor-alpha chain and STAT-6 independently of IL-5 and eotaxin. Am J Respir Cell Mol Biol, 2001. 25(4): p. 522-30.

3-15. Zhu, Z., et al., Pulmonary expression of interleukin-13 causes inflammation, mucus hypersecretion, subepithelialfibrosis, physiologic abnormalities, and eotaxin production. J Clin Invest, 1999. 103(6): p. 779-88.

3-16. Wills-Karp, M., et al., Interleukin-13: central mediator of allergic asthma. Science, 1998. 282(5397): p. 2258-61.

3-17. Grunig, G., et al., Requirement for IL-13 independently of IL-4 in experimental asthma. Science, 1998. 282(5397): p. 2261-3.

18. Elias, J. A., et al., Airway remodeling in asthma. J Clin Invest, 1999. 104(8): p. 1001-6.

3-19. Kumar, R. K., Understanding airway wall remodeling in asthma: a basis for improvements in therapy? Pharmacol Ther, 2001. 91(2): p. 93-104.

3-20. Holgate, S. T., et al., Bronchial epithelium as a key regulator of airway allergen sensitization and remodeling in asthma. Am J Respir Crit Care Med, 2000. 162(3 Pt 2): p. S113-7.

3-21. Walter, D. M., et al., Critical role for IL-13 in the development of allergen-induced airway hyperreactivity. J Immunol, 2001. 167(8): p. 4668-75.

3-22. Webb, D. C., et al., Integrated signals between IL-13, IL-4, and IL-5 regulate airways hyperreactivity. J Immunol, 2000. 165(1): p. 108-13.

3-23. Foster, P. S., et al., Dissociation of T helper type 2 cytokine-dependent airway lesions from signal transducer and activator of transcription 6 signalling in experimental chronic asthma. Clin Exp Allergy, 2003. 33(5): p. 688-95.

3-24. Yang, G., et al., A monoclonal antibody to mouse IL-13 inhibits acute asthma response. The FASEB Journal, 2002. 16(4): p. A672.

3-25. Laporte, J. C., et al., Direct effects of interleukin-13 on signaling pathways for physiological responses in cultured human airway smooth muscle cells. Am J Respir Crit Care Med, 2001. 164(1): p. 141-8.

3-26. Faffe, D. S., et al., IL-13 and IL-4 promote TARC release in human airway smooth muscle cells: role of IL-4 receptor genotype. Am J Physiol Lung Cell Mol Physiol, 2003. 285(4): p. L907-14.

3-27. Tliba, O., et al., IL-13 enhances agonist-evoked calcium signals and contractile responses in airway smooth muscle. Br J Pharmacol, 2003. 140(7): p. 1159-62.

3-28. Defrance, T., et al., Interleukin 13 is a B cell stimulating factor. J Exp Med, 1994. 179(1): p. 135-43.

3-29. Li, L., et al., Effects of Th2 cytokines on chemokine expression in the lung: IL-13 potently induces eotaxin expression by airway epithelial cells. J Immunol, 1999. 162(5): p. 2477-87.

3-30. Kumar, R. K., et al., Role of interleukin-13 in eosinophil accumulation and airway remodelling in a mouse model of chronic asthma. Clin Exp Allergy, 2002. 32(7): p. 1104-11.

3-31. Blease, K., et al., Therapeutic effect of IL-13 immunoneutralization during chronic experimental fungal asthma. J Immunol, 2001. 166(8): p. 5219-24.

3-32. Rubin, B. K., et al., Histopathology of fatal asthma: drowning in mucus. Pediatr Pulmonol, 2001. Suppl 23: p. 88-9.

3-33. Kuyper, L. M., et al., Characterization of airway plugging in fatal asthma. Am J Med, 2003. 115(1): p. 6-11.

3-34. Kondo, M., et al., Interleukin-13 induces goblet cell differentiation in primary cell culture from Guinea pig tracheal epithelium. Am J Respir Cell Mol Biol, 2002. 27(5): p. 536-41.

3-35. Homer, R. J. and J. A. Elias, Consequences of long-term inflammation. Airway remodeling. Clin Chest Med, 2000. 21(2): p. 331-43, ix.

3-36. Lee, C. G., et al., Interleukin-13 induces tissue fibrosis by selectively stimulating and activating transforming growth factor beta(1). J Exp Med, 2001. 194(6): p. 809-21.

3-37. Wenzel, S. E., et al., TGF-beta and IL-13 synergistically increase eotaxin-1 production in human airway fibroblasts. J Immunol, 2002. 169(8): p. 4613-9.

3-38. Doucet, C., et al., IL-4 and IL-13 specifically increase adhesion molecule and inflammatory cytokine expression in human lung fibroblasts. Int Immunol, 1998. 10(10): p. 1421-33.

3-39. Ingram, J. L., et al., Interleukin-13 stimulates the proliferation of lung myofibroblasts via a signal transducer and activator of transcription-6-dependent mechanism: a possible mechanism for the development of airway fibrosis in asthma. Chest, 2003. 123(3 Suppl): p. 422S-4S.

3-40. Oriente, A., et al., Interleukin-13 modulates collagen homeostasis in human skin and keloid fibroblasts. J Pharmacol Exp Ther, 2000. 292(3): p. 988-94.

3-41. Saito, A., et al., Potential action of IL-4 and IL-13 as fibrogenic factors on lung fibroblasts in vitro. Int Arch Allergy Immunol, 2003. 132(2): p. 168-76.

3-42. Kraft, M., et al., Decreased steroid responsiveness at night in nocturnal asthma. Is the macrophage responsible? Am J Respir Crit Care Med, 2001. 163(5): p. 1219-25.

3-43. Spahn, J. D., et al., A novel action of IL-13: induction of diminished monocyte glucocorticoid receptor-binding affinity. J Immunol, 1996. 157(6): p. 2654-9.

3-44. Kibe, A., et al., Differential regulation by glucocorticoid of interleukin-13-induced eosinophilia, hyperresponsiveness, and goblet cell hyperplasia in mouse airways. Am J Respir Crit Care Med, 2003. 167(1): p. 50-6.

3-45. Kraft, M., et al., IL-4, IL-13, and dexamethasone augment fibroblast proliferation in asthma. J Allergy Clin Immunol, 2001. 107(4): p. 602-6.

3-46. Lewis, C. C., et al., Interleukin-4 and interleukin-13 augment the proliferative effect of dexamethasone on distal lung fibroblasts. Chest, 2003. 123(3 Suppl): p. 356S.

### EXAMPLE 4

### Therapeutic Dosing with Anti-Interleukin-13 Monoclonal Antibody Inhibits Asthma Progression in Mice

Abstract

In vivo models have demonstrated that interleukin-13 (IL-13) plays an important role in asthma. However, few studies have evaluated the effect of inhibition of IL-13 on established and persistent disease. In the present study we have investigated the effect of a therapeutic dosing regimen with an anti-IL-13 monoclonal antibody (mAb) in a chronic mouse model of persistent asthma. Balb/c mice were sensitized to allergen (ovalbumin, OVA, on days 1 and 8) and challenged with OVA weekly from day 22. Anti-IL-13 mAb or vehicle dosing was initiated following two OVA challenges when disease was established. At this time, mice exhibited airway hyperresponsiveness (AHR), increased mucus production, inflammation and a trend towards increased subepithelial fibrosis compared to saline-challenged mice. Mice received four additional OVA challenges. Treatment with anti-IL-13 mAb inhibited AHR and prevented the further development of subepithelial fibrosis and progression of inflammation. Furthermore, mAb treatment reversed the mucus hyperplasia to basal levels. These effects were associated with an inhibition of cytokines, chemokines and matrix metalloproteinase-9. These data demonstrate that neutralization of IL-13 can inhibit the progression of established disease in the presence of repeated allergen exposures.

### Introduction

Asthma is characterized by the presence of reversible bronchoconstriction, increased sensitivity to specific and non-specific bronchospasmic agents and excessive mucus production. These clinical features are accompanied by an underlying pathology of inflammation and airway remodeling. The pathological changes are thought to contribute to the clinical symptoms of the disease (1, 2). Evidence to date suggests that interleukin-13 (IL-13) has an important role in asthma; it is associated with human disease and preclinical models have demonstrated that it induces many of the features associated with human asthma.

Elevated levels of IL-13 mRNA(3-6) and protein (3) have been described in human disease pathogenesis and polymorphisms in the IL-13 gene have been associated with asthma (7-9). Multiple studies have demonstrated that administration of recombinant murine (rm) IL-13 to the lungs of mice induces airway mucus hyperplasia, eosinophilia and airway hyperresponsiveness (AHR) (10-17). These effects of IL-13 are reproduced in transgenic mouse systems where IL-13 overexpression is induced in a constitutive or inducible manner (18-20). Chronic transgenic overexpression of IL-13 also induces subepithelial fibrosis and emphysema. Mice deficient in the IL-13 (and IL-4) signaling molecule signal transducer and activator of transcription 6 (STAT6) fail to develop allergen-induced AHR and mucus hyperplasia (21, 22). Finally, utilization of an IL-13-specific neutralization strategy with soluble IL-13 receptor fusion protein (sIL-13Ra2Fc) has demonstrated the pivotal role of this cytokine in experimental allergen-induced airway disease (10, 11, 23).

Current therapies for asthma provide symptomatic control but do not halt the underlying disease highlighting the unmet medical need. Patients are continually exposed to allergens, or other bronchospasmic agents, which contribute to the maintenance or progression of persistent disease. Clinically, therapeutic interventions occur in the presence of established disease. To date, experimental therapeutic-intervention studies have not investigated the effects of IL-13 neutralization on disease progression. In the present study, we used a rat monoclonal antibody (mAb) to neutralize the actions of mouse IL-13 specifically, which differs from a previous study that utilized polyclonal antibodies (24), and have initiated treatment with the mAb once disease is established. Further, we have continued mAb treatment in the presence of further allergen challenges. Our data demonstrate that anti-IL-13 mAb treatment inhibits AHR, chronic inflammation, subepithelial fibrosis and reverses mucus hyperplasia. Associated with these effects, neutralization of IL-13 inhibited the production of multiple cytokines, chemokines and the activity of matrixmetalloproteinase 9 (MMP-9). These data support the hypothesis that IL-13 is an important upstream mediator involved in asthma pathogenesis.

Methods

Mice: BALB/C female mice (6-8 weeks old, weighing 20-25 g) were from Charles Rivers Laboratories (Raleigh N.C.). All mice were maintained under specific pathogen free conditions and maintained on an OVA free diet with free access to food and water. All experimental animals used in this study were under a protocol approved by the Institutional Animal Care and Use Committee of Centocor.

Experimental Protocol

Mice were immunized intraperitoneally (I.P.) with 10 μg ovalbumin (OVA; Sigma, St. Louis, Mo.) in 100 μl of phosphate-buffered saline (PBS) mixed with the same volume of Inject Alum (Pierce, Rockford, Ill.) on day 1, and boosted in the same way on day 8. On day 22, 29, 36, 43, 50 and 52, mice received an intranasal (I.N.) challenge with 50 μl PBS or 100 μg OVA (2 mg/ml) under ketamine/xylazine anesthesia (90 and 10 mg/kg respectively; I.P.). Rat anti-mouse IL-13 mAb (C531; 500 μg/mouse) or vehicle (PBS; 200 μl/mouse) treatment was initiated on day 36 and agents were administered intravenously (I.V.) one hour prior to each I.N. challenge. Our previous in-house studies have established the in vivo efficacious dose of C531 and, no differences were observed between isotype control antibody- or vehicle-treated mice. This justified the use of the vehicle control in the present study. A subset of mice was sacrificed on day 36; these mice did not receive day 36 I.N. challenge or I.V. treatment.

Airway Hyperresponsiveness (AHR)

On day 36 and 53 AHR was measured in mice using whole body plethysmography (Buxco, Sharon, Conn.). AHR was measured by aerosolizing PBS or increasing concentrations of methacholine (10-40 mg/ml, Sigma) for 3 minutes in the chamber. AHR was expressed as enhanced pause (Penh; (25).

Bronchoalveolar Lavage (BAL)

After AHR measurements, mice were euthanized (CO2 asphyxiation), blood was collected for isotype antibody detection, and the lungs were perfused through the right ventricle with sterile PBS to remove the blood. BALs were obtained by flushing the lungs twice through the trachea with 1 ml PBS (containing 2% fetal calf serum (FCS) and 15 unit/ml heparin). Supernatants were collected after centrifugation (10 minutes; 1500 rpm) and stored at −70° C. for further analysis. BAL cells were resuspended in 1 ml PBS (containing 2% FCS) for total (using a hemacytometer) and differential cell counts (performed on eosin and methylene blue-stained cytospins).

Histology and Morphometric Analyses

Following BAL, the left lung was fixed with 10% buffered formalin under constant pressure. After fixation, lungs were dehydrated and embedded in paraffin by routine methods. Lungs were oriented in the blocks so that para-hilar sagital sections were obtained. Five micron serial sections were stained with hematoxylin and eosin (H&E), periodic acid Schiff (counterstained with hematoxylin) (PAS) or picric-Sirius Red (SR). H&E stained sections were used for general evaluation of histopathologic change and PAS for morphometric analysis of mucus inclusions and inflammatory infiltrates. SR, viewed and photographed under crossed polarized light, was used for morphometric analysis of mature collagen fibers (26). One 40× field of the para-hilar region, including the main stem bronchus and its primary and secondary branches, was photographed for each lung using a Nikon E800 equipped with plan apochromatic lenses and a Nikon DXM 1200 digital camera. Images were stored as red green blue (RGB).tiff files for morphometric analysis

For semi-quantitative analysis of mucus inclusions, inflammatory infiltrates and mature collagen, the area stained by PAS, hematoxylin or SR, was measured using Image-Pro Plus software (Media Cybermetrics, Silver Spring, Md.). Stained areas were segmented based on color and intensity from the RGB values. To calculate the % lung occupied by the material of interest, stained area was divided by the total area of lung (including blood and air space) included in the 40× field:

$$\% lung = \frac{Stained Area}{Total Lung Area} \times 100$$

To measure the intensity of SR staining, the original RGB file was converted to hue-saturation and intensity (HSI) and the intensity values extracted. The SR stained area was thresholded and the integrated optical density measured for each image.

Chemokine, Cytokine and MMP-9 Detection by ELISA

The right lungs were homogenized with 1 ml of PBS/sample and supernatants were collected following centrifugation and used for detection of chemokines, cytokines and MMP-9 (10000 rpm; 10 minutes). The levels of eotaxin, KC, mouse MCP-1 (JE), IL-13, IL-4, IL-5 and TNFα were assessed by sandwich ELISA kits following the manufacturer's instructions (R&D Systems, Minneapolis, Minn.). Active MMP-9 was detected using an ELISA (Amersham Biosciences, Piscataway, N.J.).

Statistical Analysis

Data are summarized using mean±standard error of the mean (S.E.M.). Statistical differences were determined by two-tailed t-tests when two data sets were compared, one-way analysis of variance (ANOVA) was used for multiple comparisons. The AHR data analyses for significant differences within the entire data set is described in the on-line supplement. Day 53 AHR data was analyzed by ANOVA. Statistical significance was claimed with p-values less than 0.05.

### Results

Neutralization of IL-13 Reverses Established AHR

It was important to establish the presence of airway disease at the time C531 treatment was initiated. Disease was induced in mice, following OVA sensitization, with two allergen challenges a week apart. One week after the second allergen challenge (day 36) a subset of mice was analyzed for the presence of allergic disease. AHR to methacholine challenge was observed in mice challenged with OVA compared to PBS-challenged mice. Anti-IL-13 treatment was initiated on day 36 and mice continued to receive multiple allergen challenges. Treatment with C531 inhibited AHR on day 53.

Neutralization of IL-13 Inhibits the Progressive Increase in Inflammation

Two OVA challenges also induced an increase in the numbers of eosinophils, mononuclear cells and neutrophils in the BAL on day 36. The increased airway inflammation was reflected in the lung histology by morphometry. Analysis of the BAL cell numbers and lung histology revealed that the airway and lung inflammation had progressed in day 53 OVA-challenged mice compared to day 36 mice. Treatment with C531 significantly inhibited the progressive increase in BAL eosinophilia. Increased numbers of BAL mononuclear cells were observed on day 53 compared to day 36 OVA-challenged mice and, treatment of mice with C531 resulted in significantly fewer mononuclear cells in the BAL. Allergen challenge stimulated a BAL neutrophilia that appeared to be modulated by C531 treatment but this did not reach significance. The inhibition of inflammation in the airways with C531 treatment was mirrored in the morphometric analyses of the lung histology. A significant increase in the total number of cells in the lung tissue in OVA-challenged mice on day 53 compared to day 36 mice. C531 treatment prevented this progressive increase in total lung inflammation.

Neutralization of IL-13 Reverses Excessive Mucus Production

Lung histology revealed a significant increase in mucus staining in allergen-challenged mice on day 36. Mucus was another pathological feature that progressed between day 36 and 53. Neutralization of IL-13 with C531 significantly inhibited not only the progressive increase in mucus from day 36 to day 53 but, reversed the mucus levels to near-background levels when compared to OVA-challenged day 36 mice.

Neutralization of IL-13 Inhibits the Development of Fibrosis

Subepithelial fibrosis was assessed by staining the lung sections with Sirius red and measuring birefringence, a measurement of the amount of mature collagen. Although an increase in subepithelial fibrosis was apparent on day 36, this did not reach significance.

Significant subepithelial fibrosis had developed by day 53 in the lungs of mice challenged with OVA. Treatment with C531 prevented the development of fibrosis.

IL-13 Regulates MMP-9 Activity and Production of Pro-Inflammatory Mediators

Airway remodeling is associated with MMP activity. To address whether IL-13 was involved in MMP activation, MMP-9 activity was measured in the lung tissue of mice. Significant increases in MMP-9 activity were observed in OVA-challenged mice on day 53. Neutralization of IL-13 resulted in inhibition of MMP-9 activity. A number of cytokines and chemokines were also modulated with anti-IL-13 treatment. Allergen challenges induced significant increases in the levels of IL-4,-5,-13, TNFα, KC and eotaxin by day 36 and these levels remained elevated on day 53 together with significant increases in MCP-1 levels (Table 1). Eotaxin levels increased further on day 53 compared to levels present on day 36. This may reflect the progressive increase in eosinophils in the BAL between the two time points.

Neutralization of IL-13 inhibited the levels of all cytokines and chemokines. The effect of C531 on IL-13 protein levels was not assessed since C531 interfered with the ELISA assay.

### Discussion

The current study has demonstrated that an IL-13-specific neutralization approach with a mAb may be an effective therapy for the treatment of asthma. To our knowledge, this is the first demonstration that therapeutic dosing with an anti-IL-13 mAb in established, persistent, progressive disease in mice modulated lung function as well as the underlying pathologic features associated with the disease. Further, our data show that anti-IL-13 treatment halts disease progression and even reverses certain features associated with experimental asthma.

Cellular and humoral lung inflammation is a characteristic feature of asthma and in vivo models of asthma mimic this aspect of disease. Our current study demonstrated a progressive increase in lung tissue and airway inflammation (eosinophils and mononuclear cells) between days 36 and 53 that was inhibited by neutralization of IL-13. A trend towards inhibition of BAL neutrophilia was also observed. Interestingly, although multiple cytokines (including IL-13) and chemokines were elevated in the airways of OVA-challenged mice from day 36, only the level of eotaxin increased further at day 53 suggesting its association with the progressive increase in inflammation. Interestingly, IL-13 has been shown to induce eotaxin expression in epithelial cells (26, Le et al JE 1999 162:2477-2487). Anti-IL-13 mAb treatment reduced, but did not completely reverse, the levels of all the cytokines and chemokines measured. Exogenous administration (10-17) or transgenic pulmonary over-expression of IL-13 itself (18-20) induces the infiltration of neutrophils, eosinophils and mononuclear cells into the lungs of mice together with stimulating the expression of many chemokines including eotaxin, MCP-1 and KC. Synergistic actions of cytokines such as TNFα with IL-13 (17, 27, 28), together with positive feedback loops (14), may explain the incomplete inhibition of the lung inflammation by anti-IL-13 treatment. Interestingly, pulmonary-specific overxpression of IL-13 does not induce IL-4 or IL-5 expression in the lungs (18). Further, lymph node cells from OVA-sensitized and challenged IL-13 gene-deleted mice release levels of IL-4 and IL-5 that are similar to that released by wild type challenged mice (29). These data indicate that the inhibition of IL-4 and IL-5 observed in the present study and others (23) is secondary to IL-13 neutralization. Indeed, it has been shown that eotaxin stimulates eosinophils to secrete IL-4 (30). The effect of IL-13 inhibition on allergic cellular inflammation remains controversial (10, 11, 23). Data suggest that IL-13 may play a protective role in acute inflammatory settings. IL-13 has been shown to be anti-inflammatory in a guinea pig model of acute allergic inflammation (31). Further, neutralization of IL-13 resulted in an exacerbation of inflammation in acute experimental models of sepsis and acute lung injury (32, 33). The allergen challenge protocol used in the present study differs significantly from previous studies: at the time mAb treatment was initiated persistent disease and pathology was established and mice continued to receive multiple allergen challenges to establish a chronic disease state. The discrepancy in data regarding an effect on cell inflammation may be due to differences in the mouse strain used, the time point at which cell numbers were evaluated together with the chronicity of the model. Since neutralization of IL-13 reduced the levels of the eosinophil-survival cytokine IL-5 and, IL-13 itself has been shown to be a survival factor for eosinophils (34), it is possible that increased apoptosis and clearance in the absence of IL-13 contributes to the diminished cell numbers.

Multiple studies, including the present study, demonstrate a dissociation between the cellular (particularly eosinophils) inflammatory component following OVA challenge and AHR (e.g. (23, 24, 29, 35). In agreement with previous studies, we have shown that inhibition of IL-13 can almost completely inhibit AHR with a modest effect on inflammation. Similarly, exogenous administration of IL-13 to mice induces AHR that is dissociated from the inflammation induced by IL-13 (11-13, 29). The mechanisms underlying IL-13-induced AHR are not fully understood. In vitro, IL-13 is able to enhance nerve-stimulated murine jejunal smooth muscle contraction that is STAT6-dependent (36) and IL-13 also increases carbachol-induced murine and human smooth muscle contraction (37). Human smooth muscle cells express the IL-4Rα and IL-13Rα1 receptors (38) and smooth muscle mast cells express the IL-13 (39). These in vitro data suggest that IL-13 may have a direct effect on smooth muscle function. However, in vivo evidence suggests an indirect effect of IL-13 on smooth muscle constriction. Reconstitution of STAT6 specifically in lung epithelial cells of STAT6-deficient mice restored IL-13-induced AHR (22) indicating that IL-13 is able to act on epithelial cells to induce AHR. Further, it has been shown that IL-13-induced AHR is partly mediated by leukotrienes (15). A recent study has demonstrated that AHR persists for up to 8 weeks, following the last OVA challenge in a chronic OVA-challenge model, in the absence of cellular inflammation and IL-13 (40). It is suggested that airway remodeling contributes towards the sustained AHR. Since IL-13 can induce multiple features of airway remodeling (discussed below) it will be interesting to evaluate whether inhibition of IL-13 during the OVA challenges will impact the ensuing persistent AHR.

Airway remodeling is a characteristic underlying feature of asthma and is thought to contribute to the symptoms associated with asthma. The features of airway remodeling assessed in the current study were goblet cell hyperplasia (measured indirectly by staining for mucus positive cells) and subepithelial fibrosis. Neutralization of IL-13 reversed the mucus hyperproduction and inhibited the development of subepithelial fibrosis. Previous studies have demonstrated that IL-13 induced mucus (hyperplasia) in vivo is partially dependent on inflammation (13, 41). IL-13 induces mucus gene expression and protein (13, 22, 42-44) that may be mediated through the epidermal growth factor receptor (41). IL-13-induced goblet cell hyperplasia is not due to cell proliferation in vivo (15) but may be due to differentiation of epithelial cells into secretory cells (42, 43, 45). IL-13 stimulates fibroblast proliferation in vitro (46-49) and in vivo (14). IL-13 may activate fibroblast directly through its receptor which is present on fibroblasts (48, 50, 51) or indirectly via the stimulation of secondary mediators such as leukotrienes, transforming growth factor-α and platelet derived growth factor (15, 47, 52, 53). IL-13 also stimulates collagen deposition from fibroblasts in vitro and in vivo (18, 50) which may by mediated through activation of TGFβ by MMP-9 (54). Interestingly, in our study, we observed a progressive increase in MMP-9 activity in the lung tissue between day 36 and 53, which was associated with the development of subepithelial fibrosis, and was inhibited by anti-IL-13 mAb. Increased levels of MMP-9 have been shown to be elevated levels of MMP-9 have been found in the airways of asthmatics (e.g. (55-57) and immunolocalization studies have demonstrated that MMP-9 expression in the subepithelial basement membrane is associated with fibrosis (57, 58).

In summary, by using a therapeutic treatment regimen with an anti-IL-13 mAb in a mouse model of persistent disease, we have demonstrated an important role of IL-13 in allergic asthma. Neutralization of IL-13 inhibited and even reversed multiple features of the pathophysiology that are inadequately controlled in the clinic at present. We believe that IL-13 neutralization has the potential to be an important therapeutic for the treatment of persistent asthma.

### Example 4 References

1. Fireman, P. 2003. Understanding asthma pathophysiology. Allergy Asthma Proc 24(2):79-83.

2. Elias, J. A., C. G. Lee, T. Zheng, B. Ma, R. J. Homer, and Z. Zhu. 2003. New insights into the pathogenesis of asthma. J Clin Invest 111(3):291-7.

3. Huang, S. K., H. Q. Xiao, J. Kleine-Tebbe, G. Paciotti, D. G. Marsh, L. M. Lichtenstein, and M. C. Liu. 1995. IL-13 expression at the sites of allergen challenge in patients with asthma. J Immunol 155(5):2688-94.

4. Humbert, M., S. R. Durham, P. Kimmitt, N. Powell, B. Assoufi, R. Pfister, G. Menz, A. B. Kay, and C. J. Corrigan. 1997. Elevated expression of messenger ribonucleic acid encoding IL-13 in the bronchial mucosa of atopic and nonatopic subjects with asthma. J Allergy Clin Immunol 99(5):657-65.

5. Kotsimbos, T. C., P. Ernst, and Q. A. Hamid. 1996. Interleukin-13 and interleukin-4 are coexpressed in atopic asthma. Proc Assoc Am Physicians 108(5):368-73.

6. Naseer, T., E. M. Minshall, D. Y. Leung, S. Laberge, P. Ernst, R. J. Martin, and Q. Hamid. 1997. Expression of IL-12 and IL-13 mRNA in asthma and their modulation in response to steroid therapy. Am J Respir Crit Care Med 155(3):845-51.

7. van der Pouw Kraan, T. C., A. van Veen, L. C. Boeije, S. A. van Tuyl, E. R. de Groot, S. O. Stapel, A. Bakker, C. L. Verweij, L. A. Aarden, and J. S. van der Zee. 1999. An IL-13 promoter polymorphism associated with increased risk of allergic asthma. Genes Immun 151(1):61-5.

8. Heinzmann, A., X. Q. Mao, M. Akaiwa, R. T. Kreomer, P. S. Gao, K. Ohshima, R. Umeshita, Y. Abe, S. Braun, T. Yamashita, M. H. Roberts, R. Sugimoto, K. Arima, Y. Arinobu, B. Yu, S. Kruse, T. Enomoto, Y. Dake, M. Kawai, S. Shimazu, S. Sasaki, C. N. Adra, M. Kitaichi, H. Inoue, K. Yamauchi, N. Tomichi, F. Kurimoto, N. Hamasaki, J. M. Hopkin, K. Izuhara, T. Shirakawa, and K. A. Deichmann. 2000. Genetic variants of IL-13 signalling and human asthma and atopy. Hum Mol Genet 9(4):549-59.

9. Howard, T. D., P. A. Whittaker, A. L. Zaiman, G. H. Koppelman, J. Xu, M. T. Hanley, D. A. Meyers, D. S. Postma, and E. R. Bleecker. 2001. Identification and association of polymorphisms in the interleukin-13 gene with asthma and atopy in a Dutch population. Am J Respir Cell Mol Biol 25(3):377-84.

10. Grunig, G., M. Warnock, A. E. Wakil, R. Venkayya, F. Brombacher, D. M. Rennick, D. Sheppard, M. Mohrs, D. D. Donaldson, R. M. Locksley, and D. B. Corry. 1998. Requirement for IL-13 independently of IL-4 in experimental asthma. Science 282(5397):2261-3.

11. Wills-Karp, M., J. Luyimbazi, X. Xu, B. Schofield, T. Y. Neben, C. L. Karp, and D. D. Donaldson. 1998. Interleukin-13: central mediator of allergic asthma. Science 282(5397):2258-61.

12. Venkayya, R., M. Lam, M. Willkom, G. Grunig, D. B. Corry, and D. J. Erle. 2002. The Th2 lymphocyte products IL-4 and IL-13 rapidly induce airway hyperresponsiveness through direct effects on resident airway cells. Am J Respir Cell Mol Biol 26(2):202-8.

13. Singer, M., J. Lefort, and B. B. Vargaftig. 2002. Granulocyte depletion and dexamethasone differentially modulate airways hyperreactivity, inflammation, mucus accumulation, and secretion induced by rmIL-13 or antigen. Am J Respir Cell Mol Biol 26(1):74-84.

14. Vargaftig, B. B., and M. Singer. 2003. Leukotrienes, IL-13, and chemokines cooperate to induce BHR and mucus in allergic mouse lungs. Am J Physiol Lung Cell Mol Physiol 284(2):L260-9.

15. Vargaftig, B. B., and M. Singer. 2003. Leukotrienes mediate murine bronchopulmonary hyperreactivity, inflammation, and part of mucosal metaplasia and tissue injury induced by recombinant murine interleukin-13. Am J Respir Cell Mol Biol 28(4):410-9.

16. Zavorotinskaya, T., A. Tomkinson, and J. E. Murphy. 2003. Treatment of experimental asthma by long-term gene therapy directed against IL-4 and IL-13. Mol Ther 7(2): 155-62.

17. Kibe, A., H. Inoue, S. Fukuyama, K. Machida, K. Matsumoto, H. Koto, T. Ikegami, H. Aizawa, and N. Hara. 2003. Differential regulation by glucocorticoid of interleukin-13-induced eosinophilia, hyperresponsiveness, and goblet cell hyperplasia in mouse airways. Am J Respir Crit Care Med 167(1):50-6.

18. Zhu, Z., R. J. Homer, Z. Wang, Q. Chen, G. P. Geba, J. Wang, Y. Zhang, and J. A. Elias. 1999. Pulmonary expression of interleukin-13 causes inflammation, mucus hypersecretion, subepithelial fibrosis, physiologic abnormalities, and eotaxin production. J Clin Invest 103(6):779-88.

19. Zhu, Z., B. Ma, R. J. Homer, T. Zheng, and J. A. Elias. 2001. Use of the tetracycline-controlled transcriptional silencer (tTS) to eliminate transgene leak in inducible overexpression transgenic mice. J Biol Chem 276(27):25222-9.

20. Lanone, S., T. Zheng, Z. Zhu, W. Liu, C. G. Lee, B. Ma, Q. Chen, R. J. Homer, J. Wang, L. A. Rabach, M. E. Rabach, J. M. Shipley, S. D. Shapiro, R. M. Senior, and J. A. Elias. 2002. Overlapping and enzyme-specific contributions of matrix metalloproteinases-9 and -12 in IL-13-induced inflammation and remodeling. J Clin Invest 110(4):463-74.

21. Kuperman, D., B. Schofield, M. Wills-Karp, and M. J. Grusby. 1998. Signal transducer and activator of transcription factor 6 (Stat6)-deficient mice are protected from antigen-induced airway hyperresponsiveness and mucus production. J Exp Med 187(6):939-48.

22. Kuperman, D. A., X. Huang, L. L. Koth, G. H. Chang, G. M. Dolganov, Z. Zhu, J. A. Elias, D. Sheppard, and D. J. Erle. 2002. Direct effects of interleukin-13 on epithelial cells cause airway hyperreactivity and mucus overproduction in asthma. Nat Med 8(8):885-9.

23. Taube, C., C. Duez, Z. H. Cui, K. Takeda, Y. H. Rha, J. W. Park, A. Balhorn, D. D. Donaldson, A. Dakhama, and E. W. Gelfand. 2002. The role of IL-13 in established allergic airway disease. J Immunol 169(11):6482-9.

24. Blease, K., C. Jakubzick, J. Westwick, N. Lukacs, S. L. Kunkel, and C. M. Hogaboam. 2001. Therapeutic effect of IL-13 immunoneutralization during chronic experimental fungal asthma. J Immunol 166(8):5219-24.

25. Hamelmann, E., J. Schwarze, K. Takeda, A. Oshiba, G. L. Larsen, C. G. Irvin, and E. W. Gelfand. 1997. Noninvasive measurement of airway responsiveness in allergic mice using barometric plethysmography. Am J Respir Crit Care Med 156(3 Pt 1):766-75.

26. Dolhnikoff, M., T. Mauad, and M. S. Ludwig. 1999. Extracellular matrix and oscillatory mechanics of rat lung parenchyma in bleomycin-induced fibrosis. Am J Respir Crit Care Med 160(5 Pt 1):1750-7.

27. Li, L., Y. Xia, A. Nguyen, Y. H. Lai, L. Feng, T. R. Mosmann, and D. Lo. 1999. Effects of Th2 cytokines on chemokine expression in the lung: IL-13 potently induces eotaxin expression by airway epithelial cells. J Immunol 162(5):2477-87.

28. Moore, P. E., T. L. Church, D. D. Chism, R. A. Panettieri, Jr., and S. A. Shore. 2002. IL-13 and IL-4 cause eotaxin release in human airway smooth muscle cells: a role for ERK. Am J Physiol Lung Cell Mol Physiol 282(4):L847-53.

29. Walter, D. M., J. J. McIntire, G. Berry, A. N. McKenzie, D. D. Donaldson, R. H. DeKruyff, and D. T. Umetsu. 2001. Critical role for IL-13 in the development of allergen-induced airway hyperreactivity. J Immunol 167(8):4668-75.

30. Bandeira-Melo, C., L. J. Woods, M. Phoofolo, and P. F. Weller. 2002. Intracrine cysteinyl leukotriene receptor-mediated signaling of eosinophil vesicular transport-mediated interleukin-4 secretion. J Exp Med 196(6):841-50.

31. Watson, M. L., A. M. White, E. M. Campbell, A. W. Smith, J. Uddin, T. Yoshimura, and J. Westwick. 1999. Anti-inflammatory actions of interleukin-13: suppression of tumor necrosis factor-alpha and antigen-induced leukocyte accumulation in the guinea pig lung. Am J Respir Cell Mol Biol 20(5):1007-12.

32. Matsukawa, A., C. M. Hogaboam, N. W. Lukacs, P. M. Lincoln, H. L. Evanoff, R. M. Strieter, and S. L. Kunkel. 2000. Expression and contribution of endogenous IL-13 in an experimental model of sepsis. J Immunol 164(5):2738-44.

33. Lentsch, A. B., B. J. Czermak, J. A. Jordan, and P. A. Ward. 1999. Regulation of acute lung inflammatory injury by endogenous IL-13. J Immunol 162(2):1071-6.

34. Horie, S., Y. Okubo, M. Hossain, E. Sato, H. Nomura, S. Koyama, J. Suzuki, M. Isobe, and M. Sekiguchi. 1997. Interleukin-13 but not interleukin-4 prolongs eosinophil survival and induces eosinophil chemotaxis. Intern Med 36(3):179-85.

35. Mattes, J., M. Yang, S. Mahalingam, J. Kuehr, D. C. Webb, L. Simson, S. P. Hogan, A. Koskinen, A. N. McKenzie, L. A. Dent, M. E. Rothenberg, K. I. Matthaei, I. G. Young, and P. S. Foster. 2002. Intrinsic defect in T cell production of interleukin (IL)-13 in the absence of both IL-5 and eotaxin precludes the development of eosinophilia and airways hyperreactivity in experimental asthma. J Exp Med 195(11):1433-44.

36. Zhao, A., J. McDermott, J. F. Urban, Jr., W. Gause, K. B. Madden, K. A. Yeung, S. C. Morris, F. D. Finkelman, and T. Shea-Donohue. 2003. Dependence of IL-4, IL-13, and nematode-induced alterations in murine small intestinal smooth muscle contractility on Stat6 and enteric nerves. J Immunol 171(2):948-54.

37. Tliba, O., D. Deshpande, H. Chen, C. Van Besien, M. Kannan, R. A. Panettieri, Jr., and Y. Amrani. 2003. IL-13 enhances agonist-evoked calcium signals and contractile responses in airway smooth muscle. Br J Pharmacol 140(7): 1159-62.

38. Laporte, J. C., P. E. Moore, S. Baraldo, M. H. Jouvin, T. L. Church, I. N. Schwartzman, R. A. Panettieri, Jr., J. P. Kinet, and S. A. Shore. 2001. Direct effects of interleukin-13 on signaling pathways for physiological responses in cultured human airway smooth muscle cells. Am J Respir Crit Care Med 164(1):141-8.

39. Brightling, C. E., F. A. Symon, S. T. Holgate, A. J. Wardlaw, I. D. Pavord, and P. Bradding. 2003. Interleukin-4 and -13 expression is co-localized to mast cells within the airway smooth muscle in asthma. Clin Exp Allergy 33(12):1711-6.

40. Leigh, R., R. Ellis, J. Wattie, D. S. Southam, M. De Hoogh, J. Gauldie, P. M. O'Byrne, and M. D. Inman. 2002. Dysfunction and remodeling of the mouse airway persist after resolution of acute allergen-induced airway inflammation. Am J Respir Cell Mol Biol 27(5):526-35.

41. Shim, J. J., K. Dabbagh, I. F. Ueki, T. Dao-Pick, P. R. Burgel, K. Takeyama, D. C. Tam, and J. A. Nadel. 2001. IL-13 induces mucin production by stimulating epidermal growth factor receptors and by activating neutrophils. Am J Physiol Lung Cell Mol Physiol 280(1):L134-40.

42. Zuhdi Alimam, M., F. M. Piazza, D. M. Selby, N. Letwin, L. Huang, and M. C. Rose. 2000. Muc-5/5ac mucin messenger RNA and protein expression is a marker of goblet cell metaplasia in murine airways. Am J Respir Cell Mol Biol 22(3):253-60.

43. Kondo, M., J. Tamaoki, K. Takeyama, J. Nakata, and A. Nagai. 2002. Interleukin-13 induces goblet cell differentiation in primary cell culture from Guinea pig tracheal epithelium. Am J Respir Cell Mol Biol 27(5):536-41.

44. Shahzeidi, S., P. K. Aujla, T. J. Nickola, Y. Chen, M. Z. Alimam, and M. C. Rose. 2003. Temporal analysis of goblet cells and mucin gene expression in murine models of allergic asthma. Exp Lung Res 29(8):549-65.

45. Laoukili, J., E. Perret, T. Willems, A. Minty, E. Parthoens, O. Houcine, A. Coste, M. Jorissen, F. Marano, D. Caput, and F. Tournier. 2001. IL-13 alters mucociliary differentiation and ciliary beating of human respiratory epithelial cells. J Clin Invest 108(12):1817-24.

46. Richter, A., S. M. Puddicombe, J. L. Lordan, F. Bucchieri, S. J. Wilson, R. Djukanovic, G. Dent, S. T. Holgate, and D. E. Davies. 2001. The contribution of interleukin (IL)-4 and IL-13 to the epithelial-mesenchymal trophic unit in asthma. Am J Respir Cell Mol Biol 25(3):385-91.

47. Ingram, J. L., A. Rice, K. Geisenhoffer, D. K. Madtes, and J. C. Bonner. 2003. Interleukin-13 stimulates the proliferation of lung myofibroblasts via a signal transducer and activator of transcription-6-dependent mechanism: a possible mechanism for the development of airway fibrosis in asthma. Chest 123(3 Suppl):422S-4S.

48. Jakubzick, C., E. S. Choi, S. L. Kunkel, B. H. Joshi, R. K. Puri, and C. M. Hogaboam. 2003. Impact of interleukin-13 responsiveness on the synthetic and proliferative properties of Th1- and Th2-type pulmonary granuloma fibroblasts. Am J Pathol 162(5):1475-86.

49. Saito, A., H. Okazaki, I. Sugawara, K. Yamamoto, and H. Takizawa. 2003. Potential action of IL-4 and IL-13 as fibrogenic factors on lung fibroblasts in vitro. Int Arch Allergy Immunol 132(2):168-76.

50. Doucet, C., D. Brouty-Boye, C. Pottin-Clemenceau, G. W. Canonica, C. Jasmin, and B. Azzarone. 1998. Interleukin (IL) 4 and IL-13 act on human lung fibroblasts. Implication in asthma. J Clin Invest 101(10):2129-39.

51. Akaiwa, M., B. Yu, R. Umeshita-Suyama, N. Terada, H. Suto, T. Koga, K. Arima, S. Matsushita, H. Saito, H. Ogawa, M. Furue, N. Hamasaki, K. Ohshima, and K. Izuhara. 2001. Localization of human interleukin 13 receptor in non-haematopoietic cells. Cytokine 13(2):75-84.

52. Booth, B. W., K. B. Adler, J. C. Bonner, F. Tournier, and L. D. Martin. 2001. Interleukin-13 induces proliferation of human airway epithelial cells in vitro via a mechanism mediated by transforming growth factor-alpha. Am J Respir Cell Mol Biol 25(6):739-43.

53. Chibana, K., Y. Ishii, T. Asakura, and T. Fukuda. 2003. Up-regulation of cysteinyl leukotriene 1 receptor by IL-13 enables human lung fibroblasts to respond to leukotriene C4 and produce eotaxin. J Immunol 170(8):4290-5.

54. Lee, C. G., R. J. Homer, Z. Zhu, S. Lanone, X. Wang, V. Koteliansky, J. M. Shipley, P. Gotwals, P. Noble, Q. Chen, R. M. Senior, and J. A. Elias. 2001. Interleukin-13 induces tissue fibrosis by selectively stimulating and activating transforming growth factor beta(1). J Exp Med 194(6):809-21.

55. Mattos, W., S. Lim, R. Russell, A. Jatakanon, K. F. Chung, and P. J. Barnes. 2002. Matrix metalloproteinase-9 expression in asthma: effect of asthma severity, allergen challenge, and inhaled corticosteroids. Chest 122(5):1543-52.

56. Han, Z., Junxu, and N. Zhong. 2003. Expression of matrix metalloproteinases MMP-9 within the airways in asthma. Respir Med 97(5):563-7.

57. Wenzel, S. E., S. Balzar, M. Cundall, and H. W. Chu. 2003. Subepithelial basement membrane immunoreactivity for matrix metalloproteinase 9: association with asthma severity, neutrophilic inflammation, and wound repair. J Allergy Clin Immunol 111(6):1345-52.

58. Hoshino, M., Y. Nakamura, J. Sim, J. Shimojo, and S. Isogai. 1998. Bronchial subepithelial fibrosis and expression of matrix metalloproteinase-9 in asthmatic airway inflammation. J Allergy Clin Immunol 102(5):783-8.

It will be clear that the invention can be practiced otherwise than as particularly described in the foregoing description and examples.

Numerous modifications and variations of the present invention are possible in light of the above teachings and, therefore, are within the scope of the appended claims.

What is claimed is:

1. A method for treating an asthma related condition in a human, comprising
(a) contacting or administering a pharmaceutical composition comprising an effective amount of at least one asthma related Ig derived protein, with, or to, said cell, tissue, organ or animal, wherein said asthma related Ig derived protein (i) inhibits at least one biological activity of interleukin-13 (IL-13) in vitro or in vivo; and (ii) specifically binds at least 1-3 amino acids of at least one selected from the group consisting of (a) 1-10, 10-20, 20-30, 30-40, 40-50, 50-60, 60-70, 70-80, 80-90, 90-100, 100-110, 110-120, 120-130, 130-140, 140-146, or 14-145 of SEQ ID NO:1.

2. A method according to claim 1, wherein said effective amount is 0.01-50 mg/kilogram of said cells, tissue, organ or animal.

3. A method according to claim 1, wherein said contacting or said administrating is by at least one mode selected from parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal.

4. A method according to claim 1, further comprising administering, prior, concurrently or after said (a) contacting or administering, at least one selected from an asthma related therapeutic, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an opthalmic, otic or nasal drug, a topical drug, a nutritional drug, a TNF antagonist, an antirheumatic, a muscle relaxant, a narcotic, an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, a cytokine, or a cytokine antagonist.

5. A method according to claim 1, wherein said asthma related condition is selected from at least one of asthma, bronchial inflammation, excess bronchial mucus or plugs, lung tissue damage, eosinophil accumulation, bronchospasm, narrowing of breathing airways, airway hypersensitivity, airway remodeling, associated pulmonary or sinus inflammation leading to at least one of inspatory or expiatory airway wheezing, breathlessness, chest tightness, coughing, dyspnea, burning, airway edema, excess mucus, bronchospasm, tachypnea, tachycardia, cyanosis, allergic rhinitis, infection, allergy; atopic dermatitis, biorhythm abnormalities, Churg-Strauss syndrome, gastroesophageal reflux disease, hay fever, and allergies.

6. A method according to claim 1, wherein said asthma related Ig derived protein is selected from an antibody, an antibody fragment, an antibody-protein fusion, a soluble receptor and a receptor fusion protein.

7. A method according to claim 1, wherein said asthma related Ig derived protein comprises at least one IL-13 binding region.

8. A method according to claim 7, wherein said IL-13 binding region comprises at least one complementarity determing region (CDR).

9. A method accoding to claim 1, wherein said asthma related Ig derived protein comprises at least a portion of at least one human heavy chain variable region or at least one light chain variable region.

10. A method according to claim 1, wherein said asthma related Ig derived protein is a substantially human Ig derived protein.

11. A method according to claim 1, wherein said asthma related Ig derived protein binds said asthma related protein with an affinity of at least one selected from at least 10⁻⁹ M, at least 10⁻¹⁰ M, at least 10⁻¹¹ M, or at least 10⁻¹² M.

12. A method according to claim 1, wherein said asthma related Ig derived protein substantially neutralizes at least one activity of at least one asthma related protein.

13. A method according to claim 4, wherein said asthma related therapeutic is selected from at least one compound or protein that acts on one or more of at least one of beta-2 agonists, anticholinergics, corticosteroids, glucocorticosteroids, anti-allergenics, anti-inflammatories, bronchiodialators, expectorants, allergy medications, cromolyn sodium, albuterol, Ventolin™, Proventil™; beclomethasone dipropionate inhaler, Vanceril™; budesonide inhaler, Pulmicort Turbuhaler™, Pulmicort Respules™; fluticasone and salmeterol oral inhaler, Advair™ Diskus; fluticasone propionate oral inhaler, Flovent™; hydrocortisone oral, Hydrocortone™, Cortef™; ipratropium bromide inhaler, Atrovent™; montelukast, Singulair™; prednisone, Deltasone™, Liquid Pred™; salmeterol, Serevent™; terbutaline, Brethine™; Bricanyl™; theophylline, Theo-Dur™, Respbid™, Slo-Bid™, Theo-24™, Theolair™, Uniphyl™, Slo-Phyllin™; triamcinolone acetonide inhaler, Azmacort™; methotrexate (MTX); interleukin antagonists such as IL-4, IL-5, IL-12 antibodies, receptor proteins or antagonists, and antagonist fusion proteins, IgE antibodies and antagonists, CD4 antagonists, antileukotrienes, platlet activating factor, thromoboxane antagonists, tryptase inhibitors, NK2 receptor antagonists, ipratropium, thephyllene, or disodium chromoglycate (DSCG).

14. A pharmaceutical composition, comprising a pharmaceutically effective amount of at least one asthma related Ig derived protein and a pharmaceutically acceptable carrier or diluent, wherein said asthma related Ig derived protein inhibits at least one biological activity of interleukin-13 (IL-13) in vitro or in vivo and specifically binds at least 1-3 amino acids of at least one selected from the group consisting of (a) 1-10, 10-20, 20-30, 30-40, 40-50, 50-60, 60-70, 70-80, 80-90, 90-100, 100-110, 110-120, 120-130, 130-140, 140-146, or 14-145 of SEQ ID NO:1.

15. A pharmaceutical composition according to claim 14, wherein said asthma related Ig derived protein is selected from an antibody, an antibody fragment, an antibody-protein fusion, a soluble receptor and a receptor fusion protein.

16. A pharmaceutical composition according to claim 1, wherein said asthma related Ig derived protein comprises at least one IL-13 binding region.

17. A pharmaceutical composition according to claim 16, wherein said IL-13 binding region comprises at least one complementarity determing region (CDR).

18. A pharmaceutical composition accoding to claim 14, wherein said asthma related Ig derived protein comprises at least a portion of at least one human heavy chain variable region or at least one light chain variable region.

19. A pharmaceutical composition according to claim 14, wherein said asthma related Ig derived protein is a substantially human Ig derived protein.

20. A pharmaceutical composition according to claim 14, wherein said asthma related Ig derived protein binds said asthma related protein with an affinity of at least one selected from at least 10⁻⁹ M, at least 10⁻¹⁰ M, at least 10⁻¹¹ M, or at least 10⁻¹² M.

21. A pharmaceutical composition according to claim 14, wherein said asthma related Ig derived protein substantially neutralizes at least one activity of at least one asthma related protein.

22. A method for treating an asthma related condition in a human, comprising
(a) contacting or administering a pharmaceutical composition comprising an effective amount of at least one asthma related Ig derived protein, with, or to, said cell, tissue, organ or animal, wherein said asthma related Ig derived protein (i) inhibits at least one biological activity of interleukin-13 (IL-13) in vitro or in vivo; and (ii) specifically binds at least 1-3 amino acids of at least one selected from the group consisting of (a) 1-10, 10-20, 20-30, 30-40, 40-50, 50-60, 60-70, 70-80, 80-90, 90-100, 100-110, 110-120, 120-130, 130-140, 140-146, or 14-145 of SEQ ID NO:1, or a mutein thereof, wherein said mutein comprises at least one substitution selected from the groups consisting of at least one of Ile48, Val48, Gln90, Glu90, Leu95, Ile95, Leu96, Ile96, Leu99, Ile99, Phe103, Tyr103, Asn130 and/or Gln130.


SEQUENCE LISTING (1 sequence)

SEQ ID NO: 1 - protein, 146 aa, Homo sapiens
MHPLLNPLLLALGLMALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN